(19) United States
(12) Patent Application Publication
(10) Pub. No.: US 2006/0009675 A1
(43) Pub. Date: Jan. 12, 2006

(54) Self-anchoring cardiac harness for treating the heart and for defibrillating and/or pacing/sensing

(76) Inventor: Steven Meyer, Oakland, CA (US)

Correspondence Address: FULWIDER PATTON, 6060 CENTER DRIVE, 10TH FLOOR, LOS ANGELES, CA, 90045, US

(21) Appl. No.: 10/931,449

(22) Filed: Sep. 1, 2004

Related U.S. Application Data
(63) Continuation-in-part of application No. 10/888,806, filed on Jul. 8, 2004.

(51) Int. Cl. A61F 13/00

(52) U.S. Cl. 600/37

(57) ABSTRACT

A self-anchoring cardiac harness is configured to fit at least a portion of a patient's heart and includes a tissue engaging element for frictionally engaging an outer surface of a heart. The engaging element produces sufficient friction relative to the outer surface of the heart, so that the harness does not migrate substantially relative to the heart. There is enough force created by the engaging element that there is no need to apply a suture to the heart in order to retain the cardiac harness. Further, the engaging element is adapted to engage the outer surface of the heart without substantially penetrating the outer surface. One or more tissue engaging elements are formed from a metal or metal alloy and are attached to a pulse generator for providing a defibrillating shock or for pacing/sensing therapy.

### CROSS-REFERENCE TO RELATED APPLICATION

This application is a continuation-in-part of U.S. Ser. No. 10/888,806 filed Jul. 8, 2004 which is incorporated herein in its entirety by reference.

### BACKGROUND OF THE INVENTION

The present invention relates to a device for treating heart failure. More specifically, the invention relates to a self-anchoring cardiac harness configured to be fit around at least a portion of a patient's heart. The cardiac harness includes an engaging element that provides a force to hold the harness onto the cardiac surface. In combination, the engaging elements hold the harness on the heart and resist migration of the harness relative to the heart during the cardiac cycle, without the need to substantially penetrate the heart.

Congestive heart failure (“CHF”) is characterized by the failure of the heart to pump blood at sufficient flow rates to meet the metabolic demand of tissues, especially the demand for oxygen. One characteristic of CHF is remodeling of at least portions of a patient's heart. Remodeling involves physical change to the size, shape and thickness of the heart wall. For example, a damaged left ventricle may have some localized thinning and stretching of a portion of the myocardium. The thinned portion of the myocardium often is functionally impaired, and other portions of the myocardium attempt to compensate. As a result, the other portions of the myocardium may expand so that the stroke volume of the ventricle is maintained notwithstanding the impaired zone of the myocardium. Such expansion may cause the left ventricle to assume a somewhat spherical shape.

Cardiac remodeling often subjects the heart wall to increased wall tension or stress, which further impairs the heart's functional performance. Often, the heart wall will dilate further in order to compensate for the impairment caused by such increased stress. Thus, a cycle can result, in which dilation leads to further dilation and greater functional impairment.

Historically, congestive heart failure has been managed with a variety of drugs. Devices have also been used to improve cardiac output. For example, left ventricular assist pumps help the heart to pump blood. Multi-chamber pacing has also been employed to optimally synchronize the beating of the heart chambers to improve cardiac output. Various skeletal muscles, such as the latissimus dorsi, have been used to assist ventricular pumping. Researchers and cardiac surgeons have also experimented with prosthetic “girdles” disposed around the heart. One such design is a prosthetic “sock” or “jacket” that is wrapped around the heart.

What has been needed, and is at this time unavailable, is a cardiac harness that resists migration off of the heart without the need to apply a suture or other attachment means to the heart or substantially penetrate the surface of the heart.

### SUMMARY OF THE INVENTION

The present invention includes a self-anchoring cardiac harness that is configured to fit at least a portion of a patient's heart and has an engaging element for frictionally engaging an outer surface of a heart. The engaging element includes at least a surface, and may include surface relief protuberances which provide a plurality of tissue engaging elements that apply respective localized forces against the heart without substantially penetrating the heart wall. Collectively, the engaging elements produce sufficient friction relative to the outer surface so that the harness does not migrate substantially relative to the outer surface. At least some of the engaging elements are formed of a metal or metal alloy that is highly conductive so that the metallic engaging elements can be used to conduct an electrical shock for defibrillation or for use in pacing/sensing therapy. The engaging elements are biocompatible and easily viewed by standard visualization processes known in the art.

In another embodiment, the self-anchoring harness can have an inner surface from which at least one grip protuberance extends. The grip protuberance includes a first surface portion lying generally in a first plane, a second surface portion lying generally in a second plane, and a peak along which the first and second surface portions meet, the peak defining an angle between the first and second planes. The peak is configured to engage a surface of's{the heart without substantially penetrating the heart surface. In one embodiment, the harness includes at least one engagement element having a plurality of grip protuberances. The engagement element can be disposed along any portion of the cardiac harness, including along elastic rows or connectors that connect adjacent rows of the harness together. In these embodiments, the grip protuberance is formed of a metal or metal alloy that is biocompatible, highly conductive, and visible under standard visualization processes known in the art.

In another embodiment, the self-anchoring cardiac harness can have at least one grip element. The grip element extends inwardly toward the heart and has a point that engages a surface of the heart without substantially penetrating the heart surface. In one embodiment, the grip element extends inwardly about 10-500 μm, and is generally conical in shape. However, the grip element may be formed into a variety of shapes, including among others, a generally pyramid-shape. A plurality of grip protuberances may be disposed on an engagement element, and the harness of the present invention may include a plurality of spaced apart engagement elements. The grip element is formed of a metal or metal alloy and is highly conductive as well.

The present invention produces friction by pressing an engaging element disposed on the cardiac harness against an outer surface of the heart. There is enough force created by the engaging element that there is no need to apply a suture or other attachment means to the heart to retain the cardiac harness. Further, the engaging elements or surface relief protuberances are adapted to engage the heart surface without substantially penetrating the heart surface.

All embodiments of the cardiac harness, including those with electrodes, are configured for delivery and implantation on the heart using minimally invasive approaches involving cardiac access through, for example, subxiphoid, subcostal, or intercostal incisions, and through the skin by percutaneous delivery using a catheter.

### BRIEF DESCRIPTION OF THE DRAWINGS

FIG. 1 depicts a schematic view of a heart with a prior art cardiac harness placed thereon.

FIGS. 2A-2B depict a spring hinge of a prior art cardiac harness in a relaxed position and under tension.

FIG. 3 depicts a perspective view of one embodiment of a cardiac harness having a plurality of rings, and tissue engaging elements disposed along the rings.

FIG. 4 depicts an unattached elongated strand or series of spring elements that are coated with a dielectric material.

FIG. 5 depicts a partial cross-sectional view of opposite ends of a ring attached to one another by a connective junction.

FIG. 6 depicts a perspective view of another embodiment of a cardiac harness having a plurality of rings, and suction cups disposed along the inner surface of the harness.

FIG. 7 depicts an enlarged partial plan view of a cardiac harness having grit disposed on the entire inner surface of the harness, including the rings of the harness and a connector that joins adjacent rings together.

FIG. 8 depicts an enlarged partial plan view of a cardiac harness having grit disposed only on a connector that joins adjacent rings together, and not on the rings of the harness.

FIG. 9 depicts an enlarged partial plan view of a cardiac harness wherein the connector is a tissue engaging element having surface relief protuberances disposed thereon.

FIG. 10 depicts an enlarged view of another embodiment of a tissue engaging element having surface relief protuberances.

FIG. 11 depicts a partial cross-sectional view of opposite ends of a ring attached to one another by a connective junction and a tissue engaging element disposed on the connective junction.

FIG. 12 depicts an enlarged view of another embodiment of a tissue engaging element disposed on a tube segment that is attached to a spring member of the cardiac harness.

FIG. 13 depicts a partial cross-section taken along line 13-13 of FIG. 10, showing the engagement element having a surface relief formed by several rows of elongated protuberances extending from a substrate.

FIG. 14 depicts an enlarged view of another embodiment of a tissue engaging element having surface relief protuberances.

FIG. 14A depicts a partial cross-section of the tissue engaging element taken along line 14A-14A of FIG. 14.

FIG. 15 depicts an enlarged view of yet another embodiment of a tissue engaging element having surface relief protuberances.

FIG. 15A depicts a partial cross-section of the tissue engaging element taken along line 15A-15A of FIG. 15.

FIG. 16 depicts an enlarged view of another embodiment of a tissue engaging element having surface relief protuberances.

FIG. 16A depicts a partial cross-section of the tissue engaging element taken along line 16A-16A of FIG. 16.

FIG. 17 depicts an enlarged view of yet another embodiment of a tissue engaging element having surface relief protuberances.

FIG. 17A depicts a partial cross-section of the tissue engaging element taken along line 17A-17A of FIG. 17.

FIG. 18 depicts a plan view of one embodiment of a tissue engaging element having a surface formed by several rows of protuberances that do not extend all the way across the engagement element.

FIG. 18A depicts a perspective view of the tissue engaging element of FIG. 18.

FIG. 19 depicts a plan view of another embodiment of a tissue engaging element having a surface formed by several rows of protuberances that are spaced apart from adjacent rows of protuberances.

FIG. 19A depicts a perspective view of the tissue engaging element of FIG. 19.

FIG. 20 depicts a plan view of an embodiment of a tissue engaging element having pyramid-shaped surface relief protuberances arranged into a row/column structure.

FIG. 20A depicts a partial cross-section of the tissue engaging element taken along ling 20A-20A of FIG. 20.

FIG. 20B depicts a partial cross-section of the tissue engaging element taken along ling 20B-20B of FIG. 20.

FIG. 21 depicts a plan view of an embodiment of a tissue engaging element having surface relief protuberances arranged into a row/column structure.

FIG. 21A depicts a partial cross-section of the tissue engaging element taken along ling 21A-21A of FIG. 21.

FIG. 21B depicts a partial cross-section of the tissue engaging element taken along ling 21B-21B of FIG. 21.

FIG. 22 depicts a perspective view of another embodiment of a tissue engaging element having surface relief protuberances with conical-shaped surfaces.

FIG. 23A depicts a plan view of an embodiment of a tissue engaging element having conical protuberances spaced apart from one another.

FIG. 23B depicts a cross-sectional view of the tissue engaging element taken along line 23B-23B of FIG. 23A.

FIG. 24 depicts a plan view of a mold for forming an array of conical protuberances.

FIG. 24A depicts a cross-sectional view of the mold taken along line 24A-24A of FIG. 24.

FIG. 25 depicts a perspective view of one embodiment of a cardiac harness having metallic tissue engaging elements disposed along the rows and associated with an ICD.

FIG. 26 depicts an enlarged partial plan view of a cardiac harness having a metallic tissue engaging element insulated from the rows of the harness.

FIG. 27 depicts an enlarged partial plan view of a cardiac harness having a metallic tissue engaging element insulated from the rows of the harness.

FIG. 28 depicts an enlarged perspective view of a metallic tissue engaging element attached to an ICD.

FIG. 29 depicts an enlarged side view of a metallic tissue engaging element electronically attached to an ICD.

### DETAILED DESCRIPTION OF THE PREFERRED EMBODIMENTS

This invention relates to a method and apparatus for treating heart failure. As discussed in Applicants' co-pending application entitled “Expandable Cardiac Harness For Treating Congestive Heart Failure”, Ser. No. 09/634,043, which was filed on Aug. 8, 2000, the entirety of which is hereby expressly incorporated by reference herein, it is anticipated that remodeling of a diseased heart can be resisted or even reversed by alleviating the wall stresses in such a heart. The present application discusses certain embodiments and methods for supporting the cardiac wall. Additional embodiments and aspects are also discussed in Applicants' co-pending applications entitled “Device for Treating Heart Failure,” Ser. No. 10/242,016, filed Sep. 10, 2002; “Heart Failure Treatment Device and Method”, Ser. No. 10/287,723, filed Oct. 31, 2002; “Method and Apparatus for Supporting a Heart”, Ser. No. 10/338,934, filed Jan. 7, 2003; and “Method and Apparatus for Treating Heart Failure,” Ser. No. 60/409,113, filed Sep. 5, 2002; “Cardiac Harness Delivery Device and Method,” Ser. No. 60/427,079, filed Nov. 15, 2002; and “Multi-panel Cardiac Harness, Ser. No. 60/458,991, filed Mar. 28, 2003, the entirety of each of which is hereby expressly incorporated by reference.

The present invention is directed to a cardiac harness system for treating the heart. The cardiac harness system of the present invention couples a cardiac harness for treating the heart coupled with a cardiac rhythm management device. More particularly, the cardiac harness includes rows or undulating strands of spring elements that provide a compressive force on the heart during diastole and systole in order to relieve wall stress pressure on the heart. Associated with the cardiac harness is a cardiac rhythm management device for treating any number of irregularities in heart beat due to, among other reasons, congestive heart failure. Thus, the cardiac rhythm management device associated with the cardiac harness can include one or more of the following: an implantable cardioverter/defibrillator with associated leads and electrodes; a cardiac pacemaker including leads and electrodes used for sensing cardiac function and providing pacing stimuli to treat synchrony of both vessels; and a combined implantable cardioverter/defibrillator and pacemaker, with associated leads and electrodes to provide a defibrillation shock and/or pacing/sensing functions.

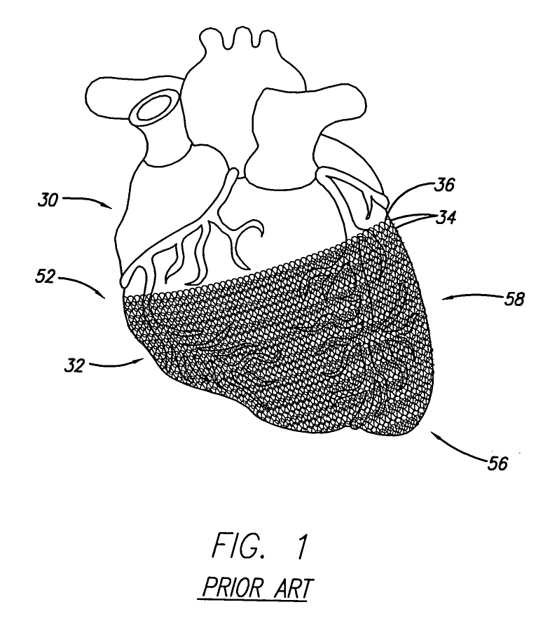

FIG. 1 illustrates a mammalian heart 30 having a prior art cardiac wall stress reduction device in the form of a harness 32 applied to it. The cardiac harness has rows 34 of elastic members 36 that circumscribe the heart and, collectively, apply a mild compressive force on the heart so as to alleviate wall stresses.

The term “cardiac harness” as used herein is a broad term that refers to a device fit onto a patient's heart to apply a compressive force on the heart during at least a portion of the cardiac cycle. A device that is intended to be fit onto and reinforce a heart and which may be referred to in the art as a “girdle,” “sock,” “jacket,” “cardiac reinforcement device,” or the like is included within the meaning of “cardiac harness.”

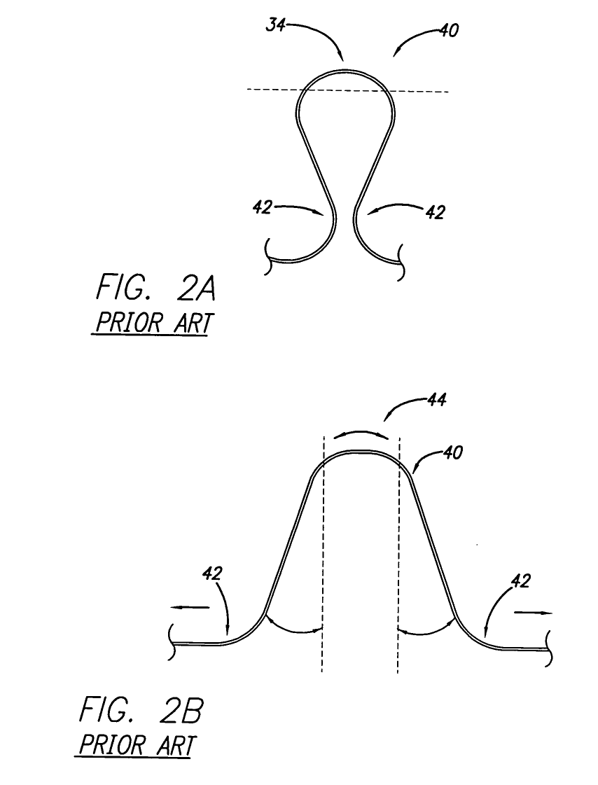

The cardiac harness 32 illustrated in FIG. 1 has several rows 34 of elastic members 36. Each row includes a series of spring elements, referred to as hinges, or spring hinges, that are configured to deform as the heart 30 expands during filling. For example, FIG. 2A shows a prior art hinge member 36 at rest. The hinge member has a central portion 40 and a pair of arms 42. As the arms are pulled, as shown in FIG. 2B, a bending moment 44 is imposed on the central portion. The bending moment urges the hinge member back to its relaxed condition. Note that a typical row or strand comprises a series of such hinges, and that the hinges are adapted to elastically expand and retract in the direction of the strand.

In the harness illustrated in FIG. 1, the elastic rows 34 are constructed of extruded wire that is deformed to form the spring elements 36.

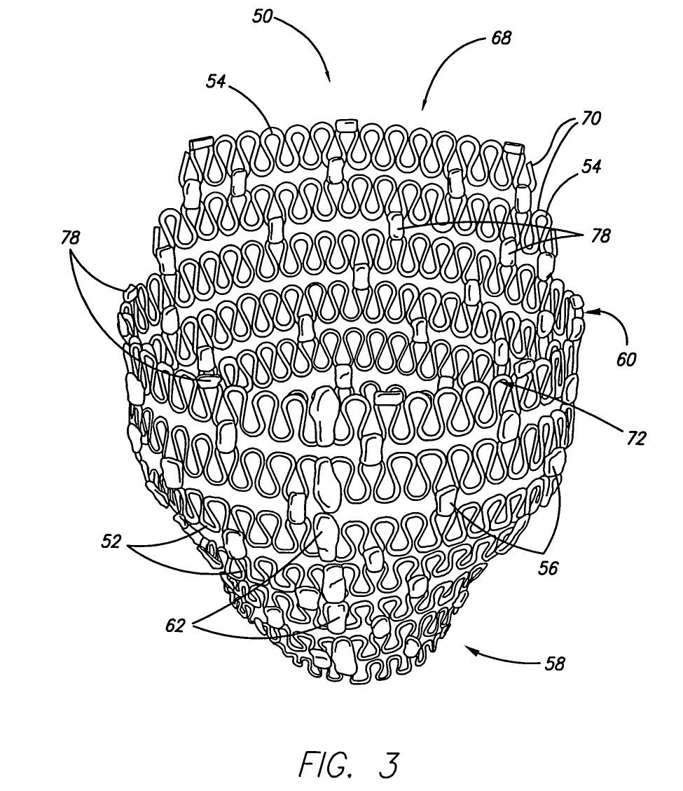

In one embodiment of the invention, as shown in FIG. 3, a cardiac harness 50 has several adjacent elastic rows 52 of spring members 54 is illustrated. In this embodiment, adjacent rows preferably are connected to one another by one or more connectors 56. The connectors help maintain the position of the elastic rows relative to one another. Preferably, the connectors have a length oriented longitudinally relative to the elastic rows so as to create a space between adjacent rows. The illustrated harness is configured to circumferentially surround at least a portion of the heart between an apex portion 58 and a base portion 60. Preferably, the connectors allow some relative movement between adjacent rows.

The connectors 56 preferably are formed of a semi-compliant material such as silicone rubber. Most preferably the connectors are formed of the same material used for coating the rings with a dielectric coating, if applicable. Some materials that can be used for the connectors include, for example, medical grade polymers such as, but not limited to, polyethylene, polypropylene, polyurethane and nylon.

As discussed above, and as discussed in more detail in the applications that are incorporated herein by reference, the elastic rows 52 exert a force in resistance to expansion of the heart. Collectively, the force exerted by the elastic rows tends toward compressing the heart, thus alleviating wall stresses in the heart as the heart expands. Accordingly, the harness helps to decrease the workload of the heart, enabling the heart to more effectively pump blood through the patient's body and enabling the heart an opportunity to heal itself. It should be understood that several arrangements and configurations of elastic rows can be used to create a mildly compressive force on the heart so as to reduce wall stresses. For example, elastic members 54 can be disposed over only a portion of the circumference of the heart or harness.

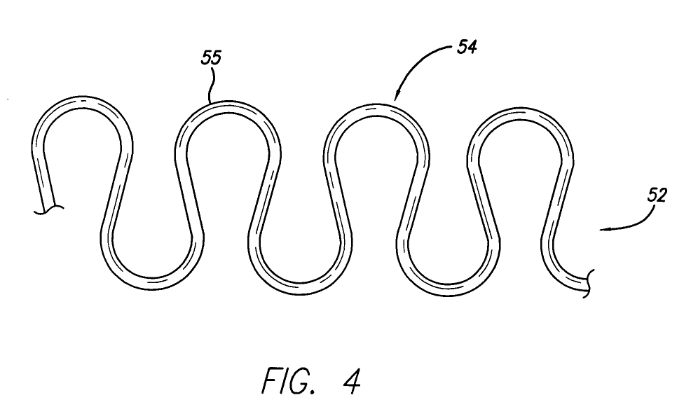

With next reference to FIG. 4, a close-up of a portion of one embodiment of an elastic row 52 is shown. In the illustrated embodiment, the row has an undulating strand of extruded wire formed into a series of successive spring elements 54. A dielectric coating 55 is disposed over the spring elements to electrically insulate the strand of extruded wire. In the illustrated embodiment, the dielectric coating includes silicone rubber. Other acceptable materials include urethanes as well as various polymers, elastomers and the like. In the illustrated embodiment, the silicone rubber coating is a tubing that has been pulled over the wire. It is to be understood that other methods for applying a coating, such as dip coating and spraying, can also be used to apply a coating to the elastic row. Further, it should be understood that in other embodiments no coating is applied over the elastic row.

In one embodiment, each elastic row 52 initially includes an elongate strand. During manufacturing of the cardiac harness 50, each elongate strand is cut to a length such that when opposite ends of the elongate strand are bonded together, the elongate strand assumes a ring-shaped configuration. The rings form the adjacent elastic rows. The lengths of the elongate strands are selected such that the resulting rings/rows are sized in conformity with the general anatomy of the patient's heart. More specifically, strands used to form the apex portion 58 of the harness are not as long as strands used to form the base portion 60. As such, the harness generally tapers from the base toward the apex in order to generally follow the shape of the patient's heart.

In another embodiment, the diameter of a ring at the base of the harness is smaller than the diameter of the adjacent ring. In this embodiment, the harness has a greatest diameter at a point between the base and apex ends, and tapers from that point to both the base and apex ends. Preferably, the point of greatest diameter is closer to the base end than to the apex end. It is contemplated that the lengths of the strands, as well as the sizes of the spring members, may be selected according to the intended size of the cardiac harness and/or the amount of compressive force the harness is intended to impart to the patient's heart.

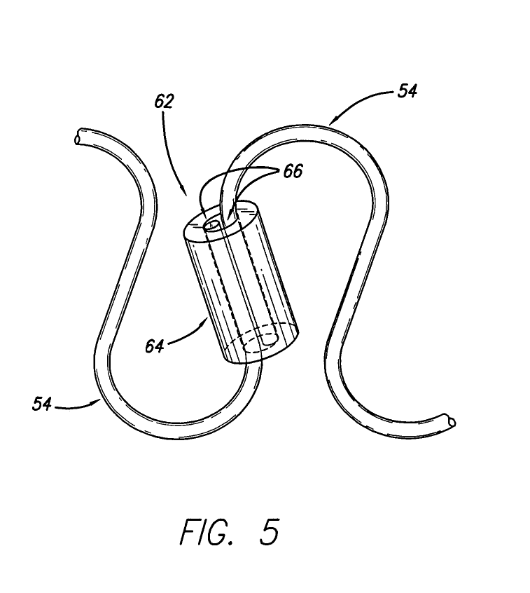

With continued reference to FIG. 3, the opposite ends of each circumferentially extending ring 52 are attached to one another by a connective junction 62. In one embodiment, illustrated in FIG. 5, each connective junction includes a small tube segment 64 into which opposite ends 66 of the ring are inserted. The tube segment serves to prevent the opposite ends of the ring from tearing loose from one another after the harness is placed on the heart. Preferably, each tube segment is filled with a dielectric material such as silicone rubber or another similar material after the ring-ends are placed therein. It is to be understood that additional methods and structure can be used to form the connective junctures. For example, the ends of the strands can be welded together or intertwined. Also, in other embodiments, each ring can be unitarily formed, such as by molding, without requiring cutting and joining of the ends.

In a human heart the right ventricle extends further from the apex of the heart than does the left ventricle. The cardiac harness 50 illustrated in FIG. 3 has a right ventricle engagement portion 68 configured to fit about the uppermost portion of the right ventricle where the ventricle begins to curve inwardly. With continued reference to FIG. 3, the right ventricle engagement portion of the harness has elastic rows that form only a partial circle. Preferably, these partial rings 70 are connected to the adjacent full ring in a manner so that the partial rings are at least mildly stretched when the rest of the harness is at rest. As such, the partial strands are biased inwardly. When placed on the heart, the partial rings “cup” the upper portion of the right ventricle. As such, the harness fits better and is held more securely on the heart than if the right side of the harness were configured the same as the left side.

In yet another embodiment, a cardiac harness has a basal-most ring 72 that is less compliant than rings elsewhere in the harness. In one embodiment, the basal-most ring has a larger diameter wire than the wire comprising the other rings of the harness. In another embodiment, the basal-most ring has a shorter length of wire than the other rings of the harness. As such, once the cardiac harness is appropriately positioned on the heart, the basal-most ring tightly engages the heart and resists apical migration of the harness. The basal-most region of the ventricles adjacent to the AV groove undergoes less circumferential change during a cardiac cycle than does the remaining bulk of the ventricles. As such, it is contemplated that the basal-most ring will have minimal or no adverse impact on cardiac performance, or cardiac cycle dynamics. It is also to be understood that, in other embodiments, multiple rings, or a basal-most portion of the harness, may have the reduced compliance. Such reduced compliance may be obtained in any manner. For example, in one embodiment, the basal-most portion is pre-stretched relative to the rest of the harness. In another embodiment, the basal-most portion is formed of a thicker or different material than other portions of the harness.

It is to be understood that several embodiments of cardiac harnesses can be constructed and that such embodiments may have varying configurations, sizes, flexibilities, etc. As discussed in the above-referenced applications, such harnesses can be constructed from many suitable materials including various metals, woven or knitted fabrics, polymers, plastics and braided filaments, and may or may not include elastic rows. Suitable harness materials also include superelastic materials and materials that exhibit shape memory. For example, a preferred embodiment is constructed of Nitinol®. Shape memory polymers can also be employed. Such shape memory polymers can include shape memory polyurethanes or other polymers such as those containing oligo(e-caprolactone) dimethacrylate and/or poly(e-caprolactone), which are available from mnemoScience. Further, harness materials can be elastic or substantially non-elastic.

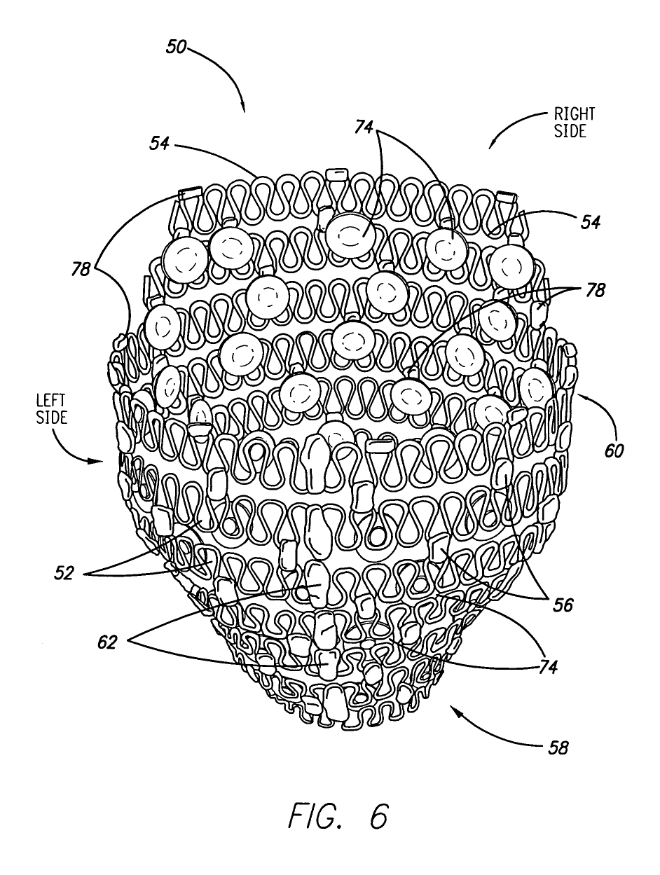

With next reference to FIG. 6, another embodiment of a cardiac harness 50 is illustrated. The illustrated harness has several inwardly-directed suction cups 74 extending from an inner surface of the harness. As shown in the illustrated embodiment, the suction cups are spaced apart from each other. Each cup is configured to engage the outer surface of the heart to create a local engagement force holding the harness onto the cardiac surface. The combined action of the several local engagement forces combine to hold the harness on the heart so as to resist migration of the harness relative to the heart during the cardiac cycle. As such, the illustrated harness embodiment anchors itself to the heart. Other embodiments of tissue engagement elements as will be described below, may also be used in conjunction with the suction cups to anchor the harness onto the heart.

In the illustrated embodiment shown in FIG. 6, the suction cups 74 may be disposed on the connectors 56 between elastic rows 52. It is to be understood, however, that in additional embodiments, suction cups can extend inwardly from any portion of the harness. In one embodiment, the suction cups are co-formed with the harness. In another embodiment, the suction cups are formed separately from the harness and are attached to the harness.

In accordance with another embodiment, a cardiac harness 50 having a structure similar to the embodiment shown and described in connection with FIG. 3 further includes a textured coating including particles or grit 76 having sizes measurable on the order of microns. As such, when the harness is disposed on the heart, and the harness gently squeezes the heart, the grit engages the heart surface so as to resist migration of the harness relative to the heart surface during the cardiac cycle.

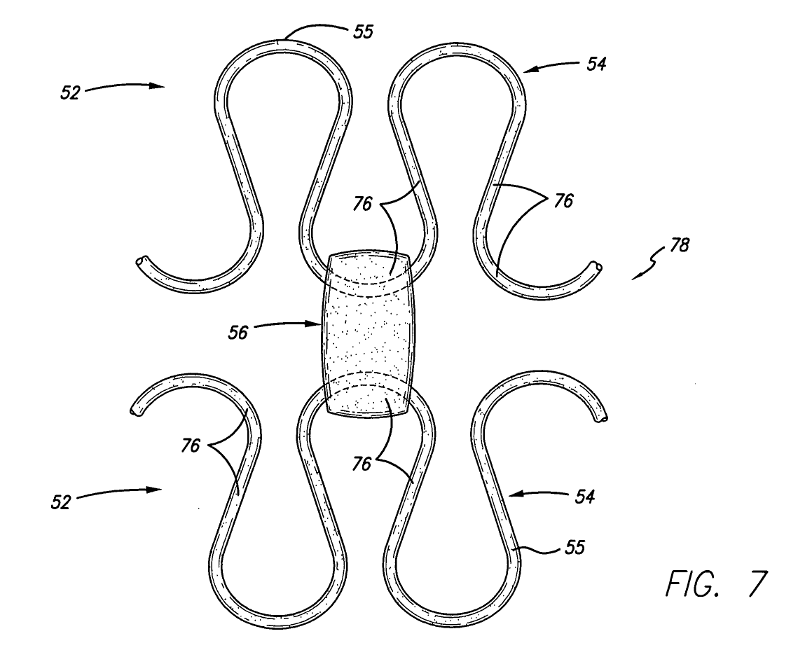

FIG. 7 is a close up view of a portion of the inner surface of a cardiac harness embodiment having a structure in accordance with this aspect. As depicted in FIG. 7, grit 76 is distributed generally around the entire inner surface of the harness. The grit may be applied to the harness in accordance with various methods such as spray coating, dipping, or the like. In the illustrated embodiment, the grit is attached to the dielectric coating 55 of the undulating wire. It is to be understood that in additional embodiments grit can be adhered directly to any structure on the inner surface of the harness.

In a preferred embodiment, a grit 76 having a size between about 10 to 500 micrometers is used. Each particle of grit, when engaged with the heart surface, creates a localized friction force that resists migration of the grit and associated harness relative to the heart surface. The several localized forces generated by each grit particle interacting with the heart surface collectively comprise a harness friction force which resists migration of the harness relative to the heart surface.

Although the grit 76 engages the heart surface and/or tissue adjacent the heart surface, it does not substantially penetrate the heart surface due to the small size of the grit particles. This should be taken to mean that the grit engaging the heart surface does not penetrate the heart surface sufficiently to cause any debilitating injury to the heart. Further, the grit does not penetrate the tissue enough to puncture any coronary vessel wall.

As discussed above, the grit 76 preferably extends from the inner wall of the cardiac harness. As such, each particle of grit includes a protuberance extending from the harness. Collectively, several particles of grit create a three-dimensional surface relief that is relatively rough and which, when engaged with the heart surface, creates a friction force that resists migration of the harness relative to the heart.

Multiple particles of grit 76, taken together, make up a tissue engagement element 78. In the embodiment illustrated in FIG. 7, since the grit is disposed generally evenly throughout the inner surface of the harness, the entire inner surface can be considered a tissue engaging element, or a certain zone or portion of the grit-covered inner surface can be defined as a tissue engagement element.

In accordance with another embodiment, a cardiac harness has a plurality of tissue engaging elements 78. Each tissue engaging element includes a surface relief made up of a plurality of protuberances. In this embodiment, surface relief protuberances are collected in tissue engaging elements, and substantially no surface relief protuberances are provided on the inner surface of the harness between tissue engagement elements, which are spaced apart from one another.

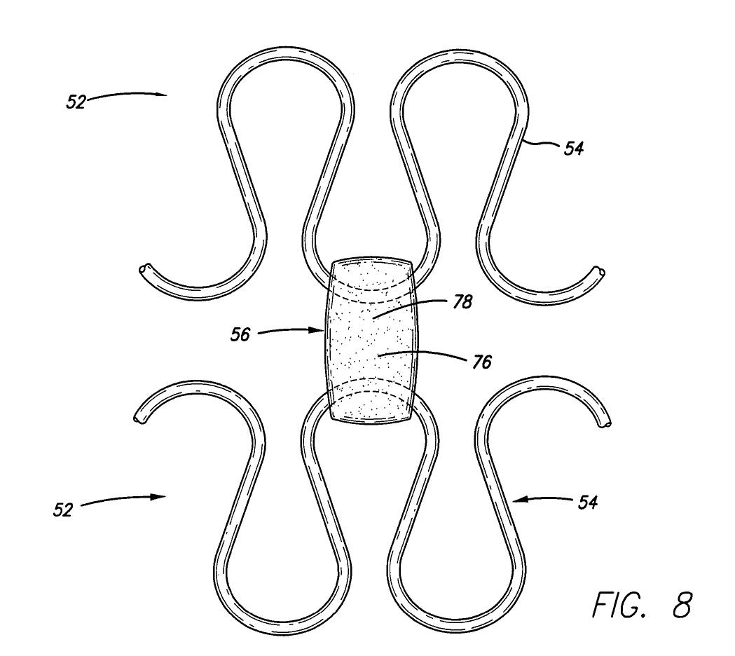

FIG. 8 shows a portion of a harness having a structure similar to the harness shown and discussed in connection with FIG. 3, wherein a plurality of tissue engagement elements 78, each having surface relief protuberances, are disposed on the inner surface of the harness and spaced apart from one another. In the illustrated embodiment, the tissue engagement elements have grit particles 76 having sizes of about 50 to 500 micrometers. More preferably the grit particles are between about 50 to 250 micrometers, still more preferably between about 60 to 200 micrometers, and most preferably between about 50 to 100 micrometers. In another embodiment, the particles are between about 200 to 400 micrometers. In a still further embodiment the grit has a medium grit of about 220 mesh. As discussed above, the grit particles have protuberances that collectively create a surface relief so that each tissue engaging element applies a localized frictional force between the heart surface and the harness in order to resist migration of the harness relative to the surface.

In the embodiments discussed above, the particles of grit preferably are sufficiently hard to engage the heart wall without bending. As such, the surface relief protuberances will firmly engage the heart wall. In a preferred embodiment, such surface relief protuberances are less compliant than the heart wall in order to ensure a thorough and firm engagement.

The grit particles 76 in the above embodiments can include any of several materials. In accordance with one embodiment, the grit particles comprise 66 μm aluminum oxide. It is to be understood that several other materials can be used. Preferably such materials include a bio-compatible material such as silica or other similarly textured materials. In another embodiment, the grit particles are biodegradable materials such as, for example, calcium sulfate, hydroxyapatite, polymethlmethacrylate (PMMA), polylactic acid (PLA), polyglycolic acid (PGA), or the like.

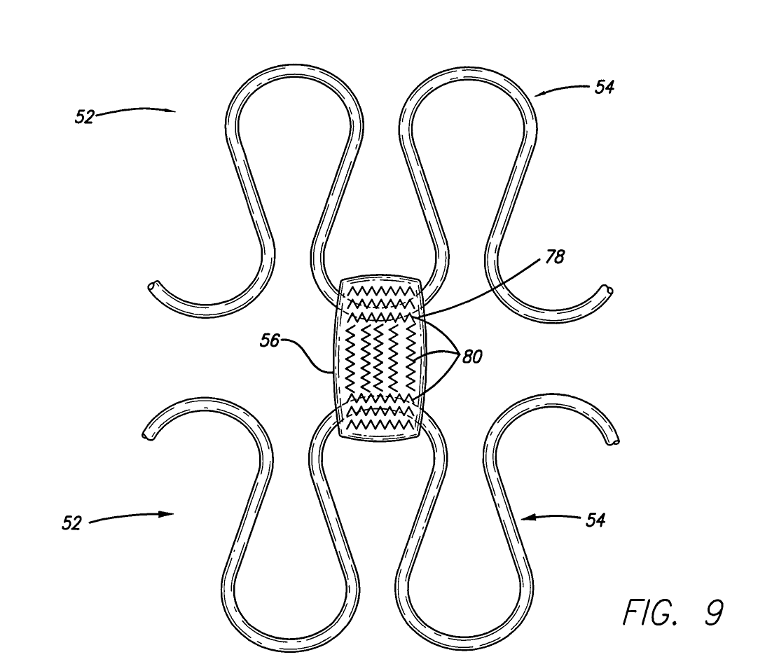

With next reference to FIG. 9, another embodiment of a cardiac harness 50 has tissue engagement elements 78 that include surface relief protuberances 80. In the illustrated embodiment, the tissue engagement element is disposed on a connector 56 between elastic rows 52. As discussed above, such connectors preferably are formed of a semi-compliant material such as, for example, silicone or urethane. In the illustrated embodiment, the inner surface of the silicone rubber connector is treated chemically in order to alter its properties, and to create surface relief protuberances that will increase the frictional force resisting relative movement between the connector and the heart surface. In accordance with one embodiment, plasma modification is used to change the cross-linking properties of the surface of the connector in order to form the tissue engaging element having surface relief protuberances. In another embodiment, other chemical processes are used to harden the surface. In another embodiment, the surface of the connector is coated with a ceramic deposition to create surface relief protuberances. In yet another embodiment, the connector is mechanically roughened such as by sanding, machining or the like in order to create surface relief protuberances. In a still further embodiment, after surface relief protuberances are formed on a connector, the surface of the connector is chemically or mechanically treated to harden the surface of the connector so that the surface relief protuberances are sufficiently rigid to engage the heart surface.

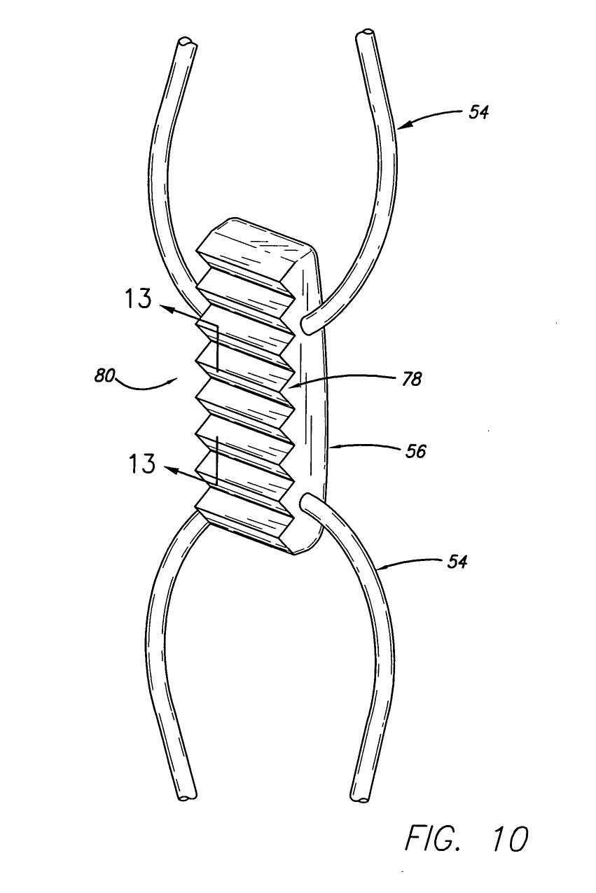

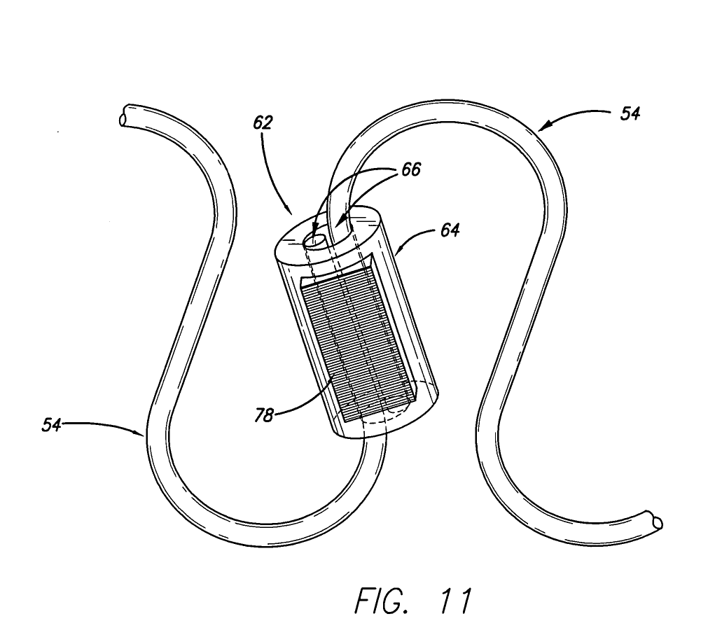

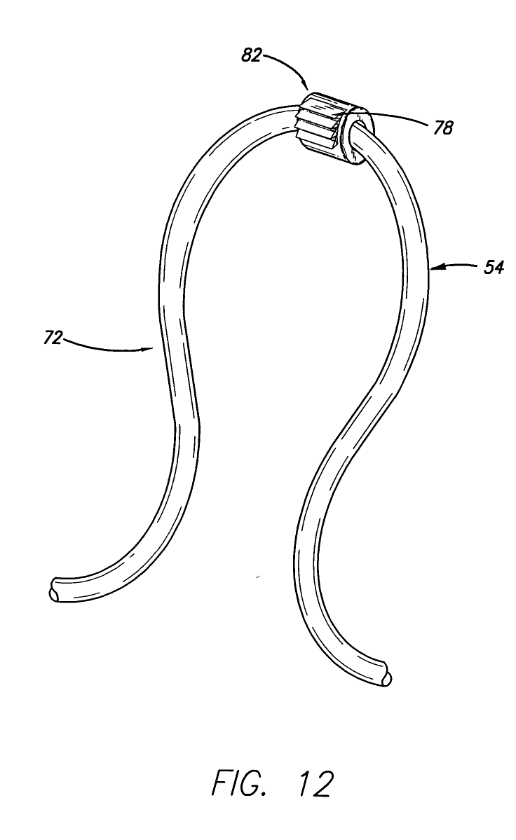

With reference next to FIG. 10, a close-up view is provided of another embodiment wherein a tissue engaging element 78 has surface relief protuberances 80 that are manufactured according to a prescribed pattern. In the illustrated embodiment, the tissue-engaging element is located on a connector 56 disposed between adjacent elastic rows 52 in an embodiment of a harness having a structure similar to that shown and described in connection with FIG. 3. With reference next to FIG. 11, in accordance with another embodiment, a tissue-engaging element 78 is disposed on a connective junction 62 of a harness. With reference next to FIG. 12, in accordance with still another embodiment, a tissue-engaging element 78 is disposed on a tube segment 82 at a basal-most ring 72 and at an upper-most portion of a harness. Each of the embodiments shown in FIGS. 10 through 12 show different arrangements of tissue-engaging elements that can be used for a harness having structure similar to that shown and described in connection with FIG. 3. It is to be understood, however, that tissue-engaging elements can be used with any cardiac harness having any type of structure.

As just discussed, an embodiment of a tissue engaging element 78 has a manufactured pattern that defines surface relief protuberances 80. It should be appreciated that several such patterns, as well as several methods and apparatus for constructing such patterns, can be employed. The discussion below presents some additional examples of tissue engaging elements.

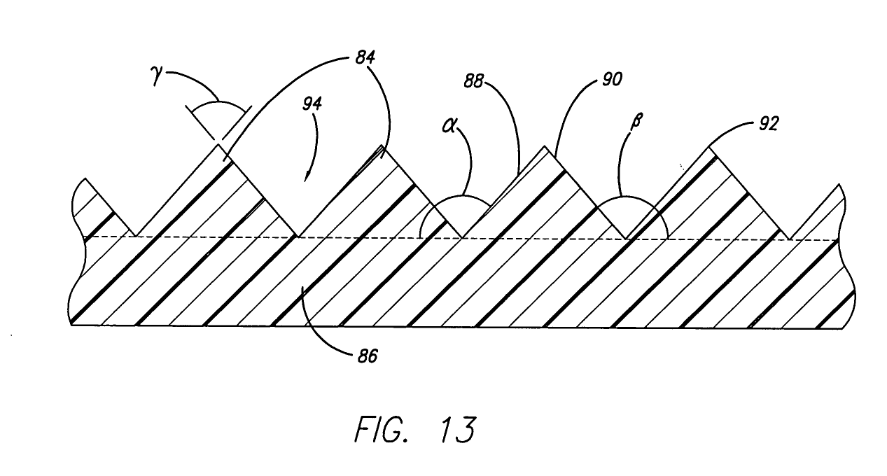

With reference again to FIG. 10 and also to FIG. 13, which is a partial cross-section of FIG. 10 taken along line 13-13, the engagement element 78 has a surface relief 80 formed by several rows of elongate protuberances 84. The protuberances extend from a substrate 86 of the engagement element. Each protuberance has a first planar surface 88 and a second planar surface 90 that intersect along an edge 91. In the illustrated embodiment, the edge also has a peak 92, which is the furthest-most point from the substrate of the engagement element. As there are several rows of protuberances, there is a space 94 between adjacent protuberance peaks.

The first planar surface 88 is disposed at a first angle α relative to a tangent or plane of the substrate 86. The first angle is measured from the open face of the first surface to the substrate. The second planar surface 90 is disposed at a second angle β. An edge or peak angle γ is defined by the intersection of the first and second planar surfaces. In the illustrated embodiment, the first and second angles are generally the same, about 135°, and the peak angle is about 90°. Of course, in other embodiments, the first and second angles are not necessarily the same, and one of the angles can be acute. Further, in other embodiments the peak angle can be acute or obtuse.

In accordance with this embodiment, the tissue engagement element 78 is configured so that the protuberances 84 engage the heart surface. Preferably, the size and peak angles γ of the protuberances are configured so that they engage heart tissue without substantially penetrating the heart surface, but also create a friction force that will resist migration of the engagement element relative to the heart surface in at least a direction generally transverse to the edge of the protuberances.

In accordance with one embodiment, material is extruded in the shape of the tissue engagement element embodiment discussed above. The extruded material is then cut to the size and shape of the engagement element 78 shown in FIG. 10. The engagement element is then bonded or otherwise attached to the harness. In the illustrated embodiment, the engagement element is bonded to a connector 56 disposed between adjacent elastic rows 52. It is to be understood that, in other embodiments, the engagement element can be adhered or otherwise attached to any portion of a cardiac harness. Additionally, in accordance with other embodiments, an engagement element can be molded, machined or otherwise formed. Further, an engagement element can be attached to a connector, or an engagement element can be co-formed as part of a connector.

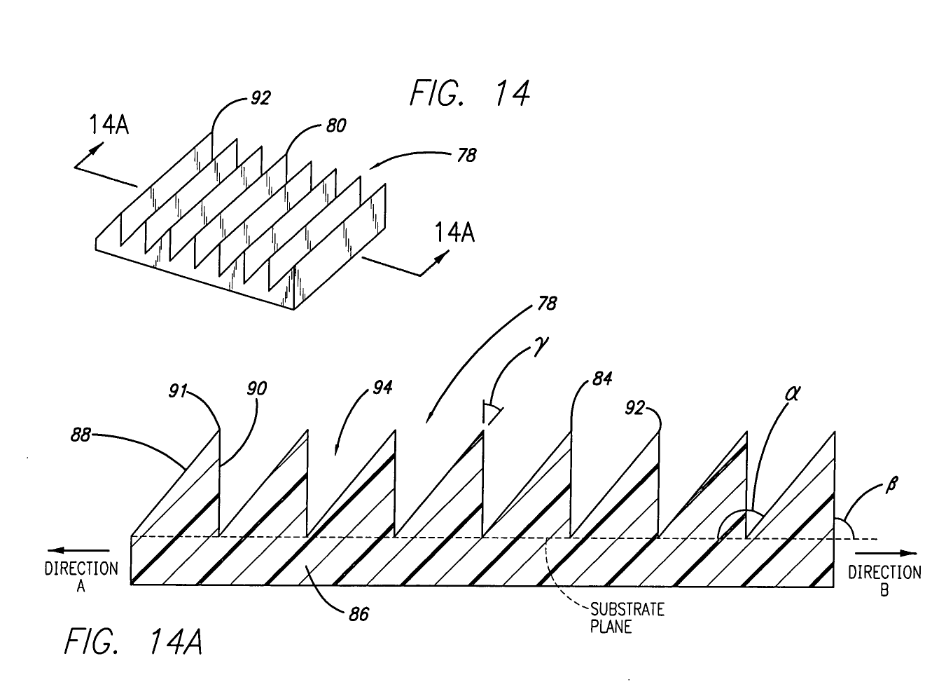

With reference to FIG. 14, a close-up view is provided of another embodiment wherein a tissue engaging element 78 has surface relief protuberances 80 that are manufactured according to a prescribed pattern. As illustrated in FIG. 14A, which is a cross-section of FIG. 14 taken along line 14A-14A, the tissue engaging element has several rows of elongate protuberances 84. The protuberances extend from a substrate 86 of the engaging element. Each protuberance has a first planar surface 88 and a second planar surface 90 that intersect along an edge 91. In the illustrated embodiment, the edge also has a peak 92, which is the furthest point from the substrate of the engaging element. There is a space 94 between adjacent protuberance peaks. When the engaging element is placed in contact with the tissue of the heart, the protuberances produce a friction force which is greatest in a direction generally transverse to the edges of the protuberances. The tissue engaging element is configured so that the protuberances engage the surface tissue of the heart without substantially penetrating the heart surface and so as to create a friction force that will resist migration of the tissue engaging element.

With continued reference to FIGS. 14 and 14A, each of the protuberances 84 may be viewed as defined by an first angle α, a second angle β, and an edge or peak angle γ. The first angle is formed by the intersection of the first planar surface 88 and a plane defined by the extent of the substrate 86. The second angle is formed by the intersection of the second planar surface 90 and the plane of the substrate. The edge angle is defined by the intersection of the first and second surfaces. In the embodiment illustrated in FIGS. 14 and 14A, the first angle is about 135 degrees, the second angle is about 90 degrees and the edge angle is about 45°. It should be understood that, in other embodiments, the first and second angles may be different. It will be appreciated that changing the size, angles and/or the spacing of the protuberances changes the level and behavior of the friction forces between the engaging element and the heart surface, and thus affects the behavior of the tissue engaging element in suppressing migration of the harness on the heart surface.

With continued references to FIGS. 14 and 14A, the first plane angle α is greater than the second plane angle β. In this arrangement, a frictional force resisting migration of the engagement element in direction B is greater than a frictional force resisting migration of the engagement element in direction A. Thus, the engagement element of FIGS. 14 and 14A exhibits preferential migration resistance in direction B.

In accordance with one embodiment, several such preferentially directional engagement elements are installed on a cardiac harness so that the harness preferentially resists migration in a direction that is generally downwardly relative to a longitudinal axis of the heart. As such, the harness will preferentially migrate upwardly toward the base of the heart. Preferably, the structure of the harness at and around the apex is configured to prevent the harness from moving too far upwardly. Simultaneously, the directional engagement elements prevent the harness from working itself downwardly over the apex and off of the heart. Thus, the harness is held snugly in place.

In another embodiment, a plurality of directional engagement elements are disposed in various orientations around the harness. Although each engagement element exhibits preferential migration resistance, the combined effect of the plurality of variously-arranged elements holds the harness in place on the heart without substantial preferential migration in any direction. In still another embodiment, directional engagement elements are disposed on the harness so that certain zones of the harness have a preferential migration resistance. Thus, certain portions of the harness will tend to migrate in a preferred direction. For example, a right side of the harness may be configured to preferentially migrate upwardly so that the harness covers a greater proportion of the right ventricle which, as discussed above, extends farther from the apex than does the left ventricle.

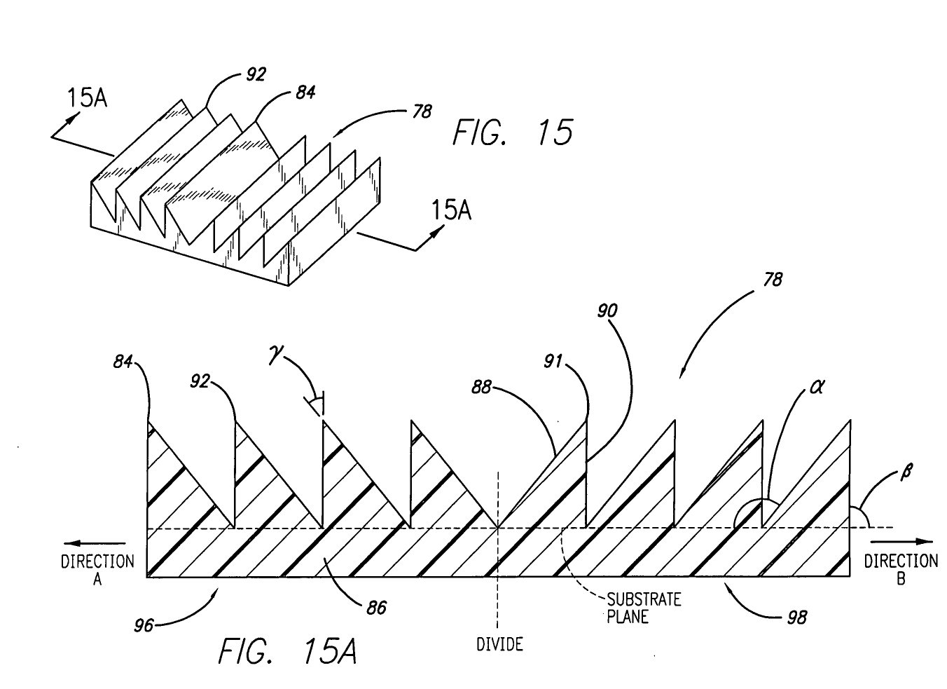

With reference next to FIGS. 15 and 15A, a close-up view is provided of another embodiment wherein a tissue engaging element 78 has surface relief protuberances 80 that are manufactured according to a prescribed pattern. The tissue engaging element shown in FIG. 15 is similar to the tissue engaging element shown in FIG. 14, except as described below. As best illustrated in FIG. 15A, which is a cross-section of FIG. 15, taken along line 15A-1SA, on a first side 96 of a dividing line of the tissue engaging element, the protuberances 84 are oriented in a first arrangement that preferentially resists movement in direction A. On a second side 98 of the dividing line of the tissue engaging element, the protuberances are oriented in a second arrangement that preferentially resists movement in direction B. It will be appreciated that because the directions A and B are opposite to one another, the engaging element produces oppositely directed friction forces on the heart surface. Thus, the tissue engaging element resists migration in both directions A and B.

In the embodiment illustrated in FIGS. 15 and 15A, the first angle α is about 90 degrees and the second angle β is about 135 degrees in the first arrangement, but the first angle is about 135° and the second angle is about 90° in the second arrangement. It is to be understood that plane angles need not be uniform throughout an engagement element and, in some embodiments adjacent protuberances may have different plane angles.

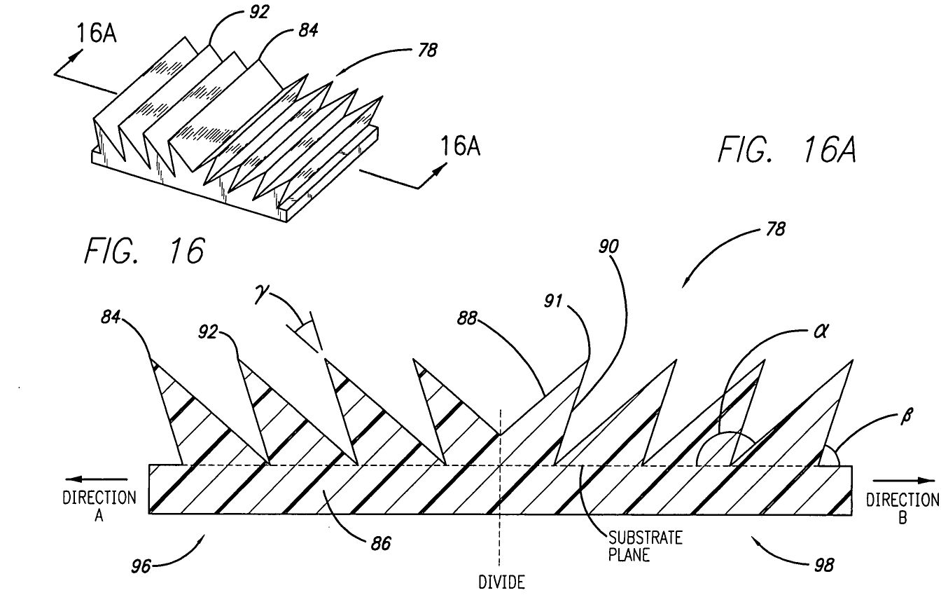

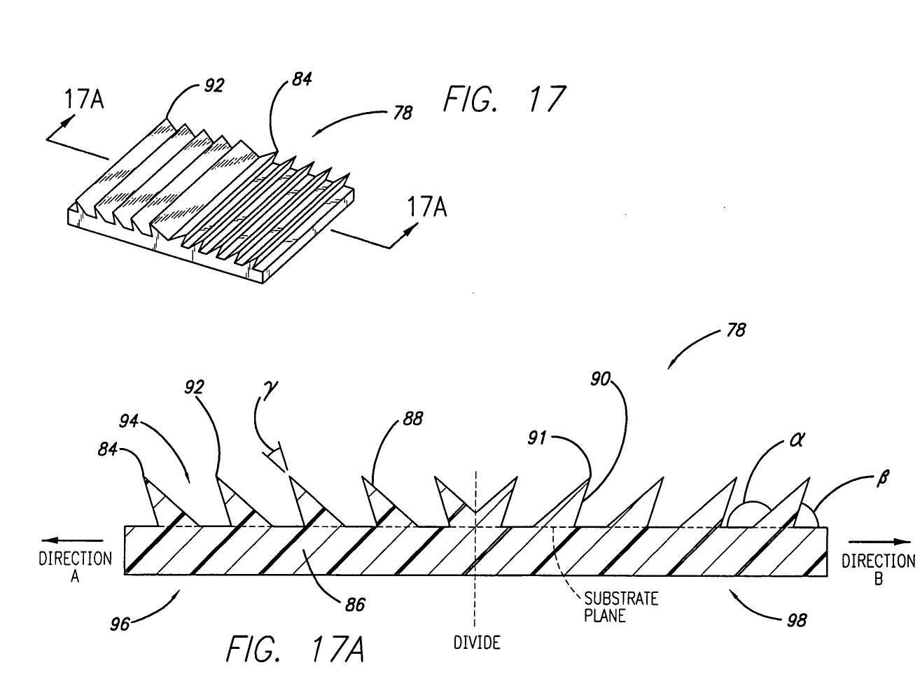

FIGS. 16 and 16A illustrate another embodiment of a tissue engaging element 78 which is capable of gripping the surface tissue of the heart. The tissue engaging element illustrated in FIGS. 16-16A, is substantially similar to the engaging element illustrated in FIGS. 15-15A. However, the plane angles α and β in FIGS. 16-16A differ from those of FIGS. 15-15A. For example, on the first side 96, the first angle is acute and the second angle is an obtuse angle of more than about 135°. A similar embodiment of a tissue engaging element 78 is illustrated in FIGS. 17 and 17A. The tissue engaging element illustrated in FIGS. 17-17A has a space 94 between adjacent protuberances 84. It will be appreciated that changing the size, angles and/or the spacing of the protuberances changes the level of the friction force which the engaging element can exert on the heart surface, and thus affects the level to which the tissue engaging element suppresses migration of the harness on the heart surface.

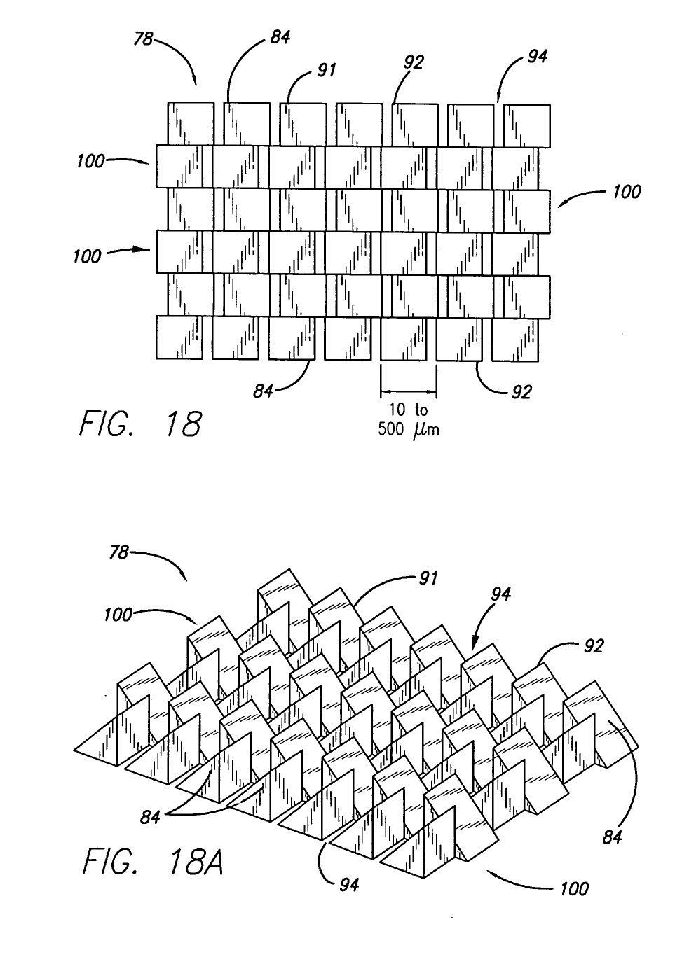

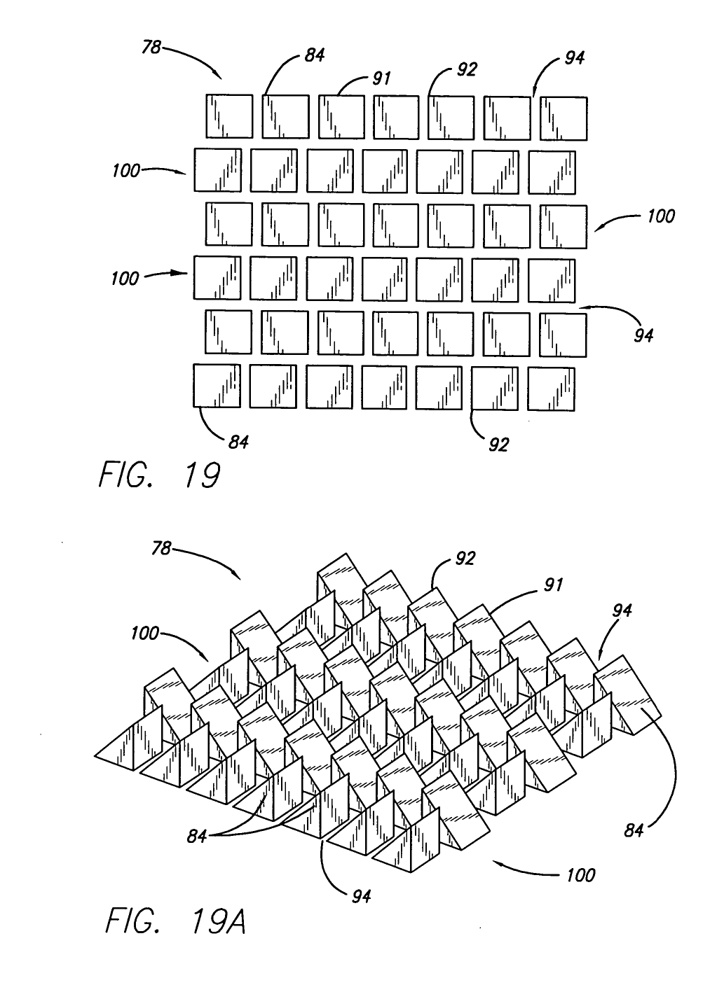

FIGS. 18 and 18A illustrate one embodiment of a tissue engaging element 78 which has a surface relief formed by several rows of protuberances 84. The protuberances illustrated in FIGS. 18-18A are substantially similar to the elongate protuberances illustrated in FIGS. 14-14A. However, the protuberances illustrated in FIGS. 18-18A do not extend all the way across the engagement element. Instead, a plurality of rows 100 of protuberances are disposed adjacent one another. As best shown in FIG. 18A, each protuberance terminates with an upper-most edge which also has a peak 92. As there are several protuberances in each row, there is a space 94 between adjacent protuberance peaks. The protuberances in each row preferably have a peak-to-peak spacing of about 10 μm to 500 μm. Each row is arranged to preferentially frictionally resist movement in one direction. Adjacent rows preferably have opposite preferred resistance directions. In other embodiments, the adjacent rows may be spaced apart from one another. For example, in the embodiment illustrated in FIGS. 19 and 19A, adjacent rows are separated by a space 94. With reference to FIGS. 18 through 19A, it will be appreciated that because adjacent rows are capable of producing friction forces in opposite directions on the heart surface, the totality of the rows forming the tissue engaging element are capable of producing friction forces which grip the surface tissue of the heart.

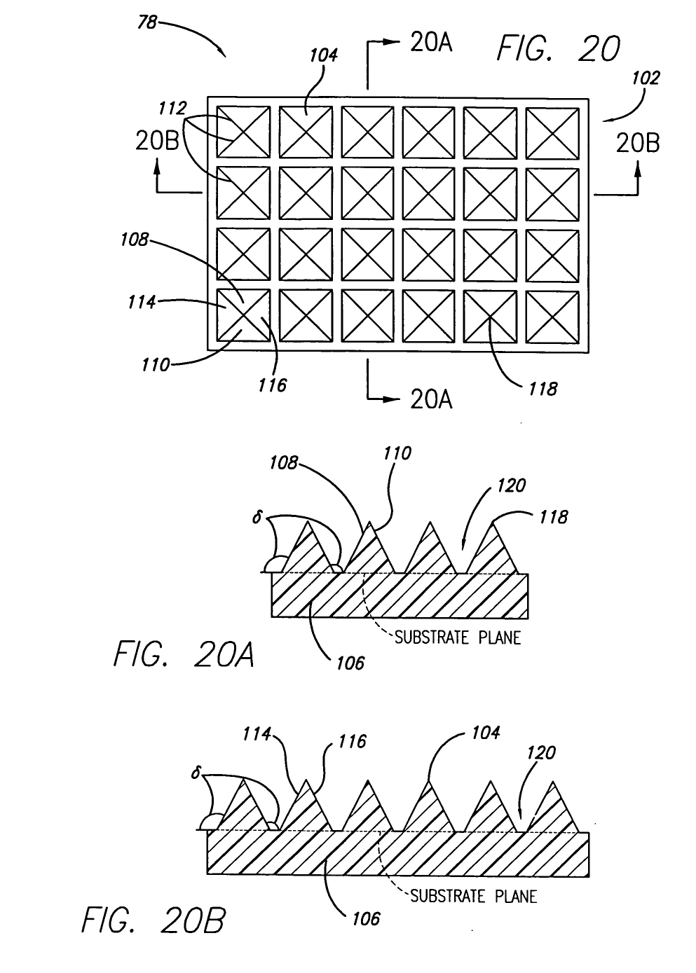

With reference next to FIG. 20, one embodiment of a tissue engaging element 78 has surface relief protuberances 102 that are arranged into a row/column structure. As shown in FIG. 20A, which is a cross-section of FIG. 20 taken along line 20A-20A, the tissue engaging element has a surface relief formed by several rows of protuberances 104. The protuberances extend from a substrate 106 of the engaging element. Each protuberance has a first planar surface 108 and a second planar surface 110 that intersect along an edge 112. Similarly, as shown in FIG. 20B, which is a cross-section of FIG. 20 taken along line 20B-20B, the surface relief protuberances of the engaging element are divided into several columns. Each protuberance comprises a third planar surface 114 and a fourth planar surface 116 that intersect along an edge. As illustrated in FIG. 20 the edges formed by the planar surfaces intersect at a peak 118, which is the furthest point from the substrate of the engaging element. In the illustrated embodiment, the peak is generally pointed, and the edges at which the planes intersect are not elongate.

With continued reference to FIG. 20, each of the planar surfaces 108, 110, 114 and 116 has an inclination angle δ. The inclination angle is formed by the intersection of the planar surface and a plane defined by the surface of the substrate. In the illustrated embodiment, the four planar surfaces have equal inclination angles, thus giving the protuberances a pyramid shape. As there are several rows and columns of protuberances, there is a space 120 between adjacent protuberance peaks. When the tissue engaging element is placed in contact with the heart surface, the protuberances engage the surface tissue without substantially penetrating the heart surface so as to create a friction force that will resist migration of the tissue engaging element relative to the heart surface.

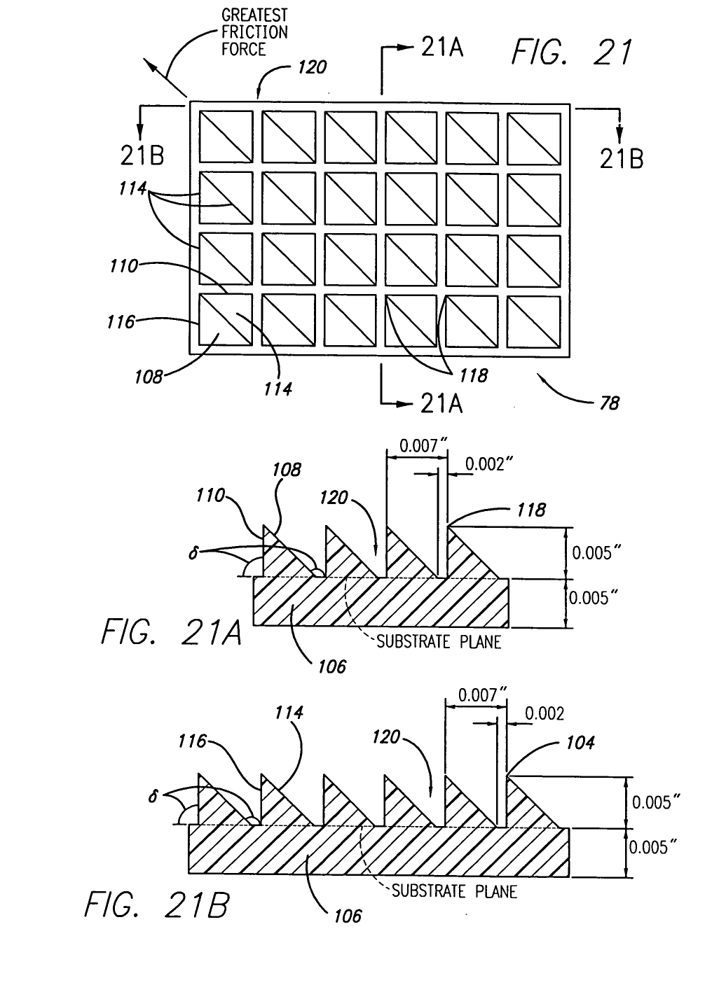

With continued reference to FIG. 20, because the planar surfaces 108, 110, 114 and 116 have the same inclination angles δ, the peaks 118 are centrally positioned within the pyramid-shaped protuberances. Thus, the tissue engaging element produces friction forces that resist migration of the harness generally equally in directions facing each plane. In another embodiment, the peaks are advantageously positioned off-center so that frictional forces resisting migration in a first direction are greater than frictional forces resisting migration is a second direction. FIG. 21 illustrates one embodiment of a tissue engaging element that has pyramid-shaped protuberances with off-center peaks.

As shown in FIG. 21A, which is a cross-section of FIG. 21 taken along line 21A-21A, the tissue engaging element 78 has a surface relief formed by several rows and columns of protuberances 104. The protuberances extend from a substrate 106 of the engaging element. Each protuberance has a first planar surface 108 and a second planar surface 110 that intersect along an edge 112. Similarly, as shown in FIG. 21B, which is a cross section of FIG. 21 taken along line 21B-21B, the surface relief protuberances are arranged into several columns that extend from the substrate of the engaging element. Each protuberance has a third planar surface 114 and a fourth planar surface 116 that intersect along an edge 117. As illustrated in FIG. 21 the edges formed by the planar surfaces intersect at a peak 118, which is the furthest point from the substrate of the engaging element. In one embodiment, the protuberances extend to a height of about 0.005 inches or less above the substrate. The peaks are separated from adjacent peaks within the same row/column by a distance of about 0.007 inches.

With continued reference to FIG. 21, each of the planar surfaces 108, 110, 114 and 116 of the protuberances 104 can be viewed as defined by an inclination angle δ. The inclination angle is formed by the intersection of the planar surface and a plane defined by the surface of the substrate 106. In the illustrated embodiment, the inclination and third planar surfaces have equal inclination angles of about 135 degrees, while the second and fourth planar surfaces have equal inclination angles of about 90 degrees. Because of the difference in inclination angles, the peaks 118 are not centrally positioned on the protuberances. Instead, the peaks are off center as shown in FIG. 21. When the tissue engaging element is placed in contact with the tissue of the heart, the off-center peaks of the protuberances engage the surface tissue of the heart so as to create friction forces that provide greater resistance to migration of the tissue engaging element in a first direction than in a second direction.

It is to be noted that in other embodiments, the inclination angles of the second and fourth planar surfaces may be greater than or lesser than about 90 degrees. Likewise, in other embodiments the inclination angles of the first and third planar surfaces may be greater than or lesser than about 135 degrees. In still other embodiments, the inclination angles of all the planar surfaces may advantageously be varied from the angles illustrated herein. It is to be further noted that although FIGS. 20 and 21 show protuberances having four planar surfaces, in other embodiments the protuberances can be comprised of more than or lesser than four planar surfaces.

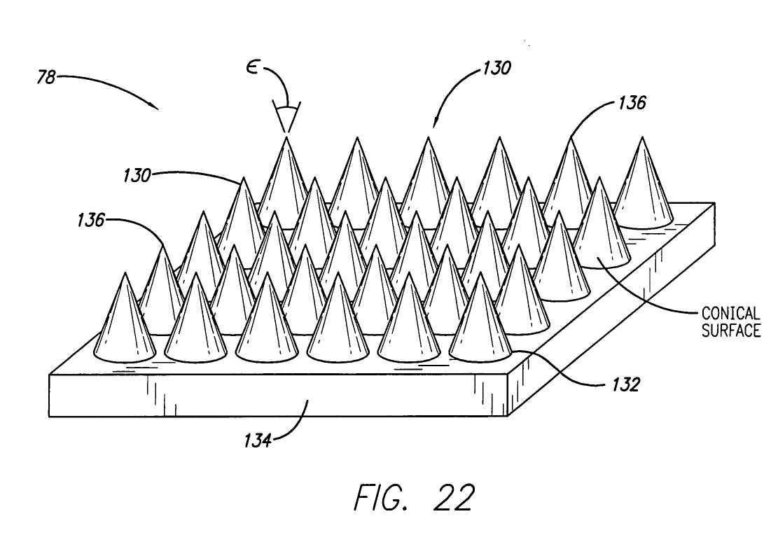

With reference next to FIG. 22, another embodiment of a tissue engaging element 78 is illustrated. The tissue engaging element shown in FIG. 22 has several rows of protuberances 130 having conical surfaces. The conical surface of each protuberance extends from a base 132 at a substrate 134 and terminates in a generally pointed peak 136. The several protuberances comprising the engaging element are arranged into a row/column structure. Of course, it is to be understood that other embodiments may not employ such a row/column structure.

With continued reference to FIG. 22, the peaks 136 of the conical protuberances 130 are centrally positioned. In one embodiment, each of the peaks has an angle ε of about 60 degrees. In other embodiments, however, the angle of the peaks may be greater than or lesser than about 60 degrees. For example, the peak angle preferably is less than about 135°. More preferably the peak angle is between about 15-115°, and more preferably is between about 30-90°. Most preferably the peak angle is between about 45-75°. In any case, the peak angle and peak height preferably are arranged so that the protuberances will not substantially penetrate the heart surface when the element is engaged with heart tissue.

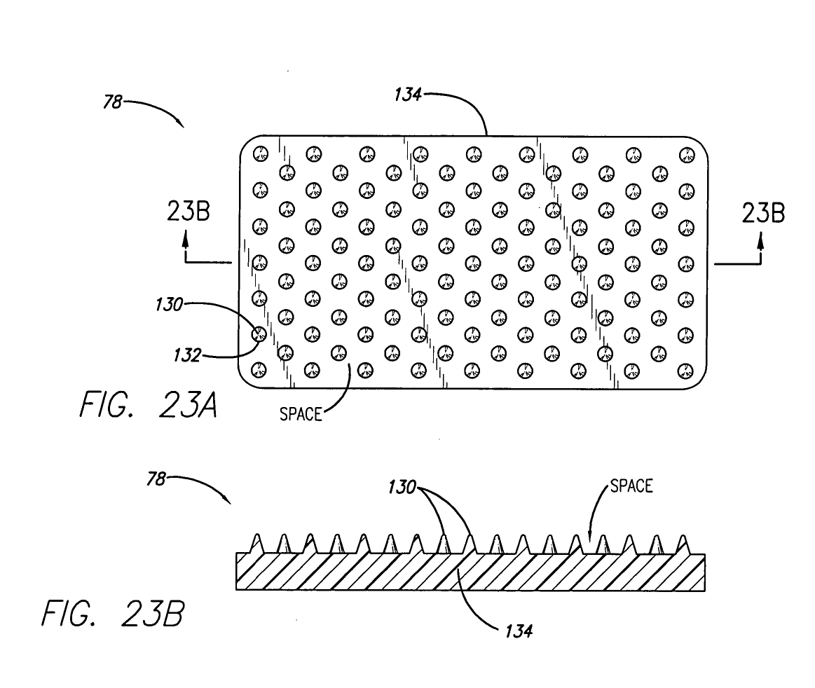

FIGS. 23A and 23B illustrate one embodiment of a tissue engaging element 78 having conical protuberances 130. In the embodiment shown in FIGS. 23A and 23B, the bases 132 of adjacent protuberances are spaced from one another.

In other embodiments, the peaks 136 of the conical protuberances 130 may be positioned off center. Thus, when the tissue engaging element is placed in contact with the tissue of the heart, the off-center peaks of the protuberances create preferential friction forces that preferentially resist migration of the tissue engaging element in at least one direction.

The tissue engaging elements disclosed herein can be manufactured by any of many processes and of many appropriate materials. Preferably, the material to be formed into the protuberances is less compliant than the heart wall so that the protuberances can effectively engage the heart wall. The protuberances preferably extend from the substrate a distance comparable to the size of the grit discussed in previous embodiments. Preferably, the protuberances extend between about 10 to 500 micrometers from the substrate. In other embodiments, the protuberances are between about 50 to 250 micrometers high, or are between about 60 to 200 micrometers. In a still further embodiment, the protuberances are between about 50 to 125 micrometers high. In yet another embodiment, the protuberances are between about 200 to 400 micrometers high.

Moreover, although the protuberances engage the heart surface, they preferably are configured so that they do not substantially penetrate the heart surface due to the size of the protuberances and the characteristics of the peak. This should be taken to mean that the protuberances engaging the heart surface do not penetrate the heart epicardium sufficient to cause debilitating injury to the heart. Further, the protuberances do not penetrate the tissue enough to puncture any coronary vessel wall.

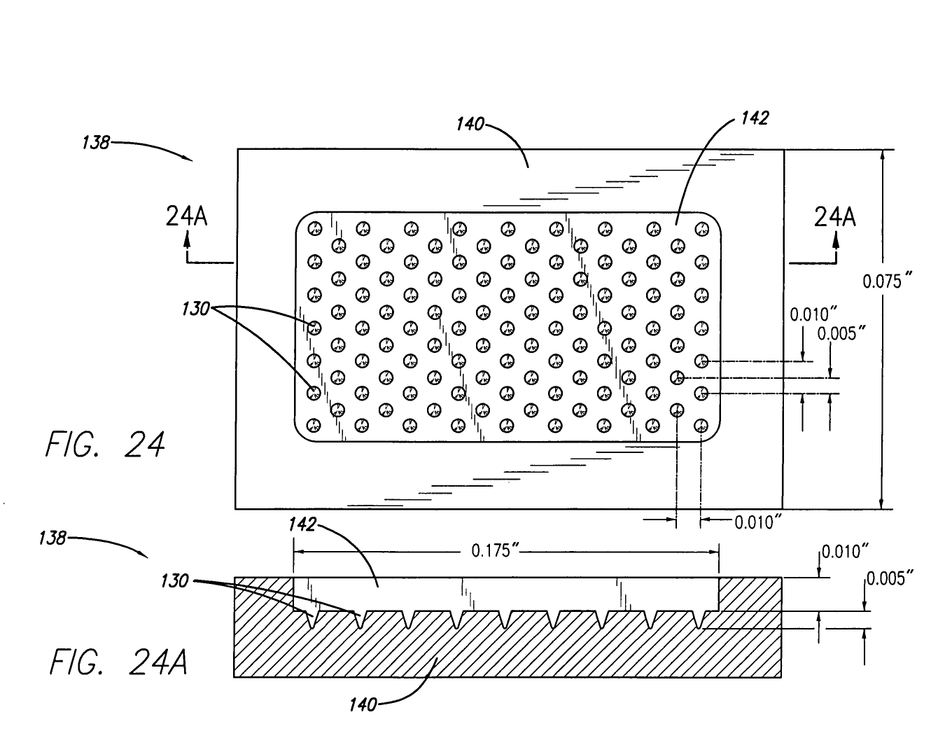

With reference to FIGS. 24 and 24A, one example of a method and apparatus for making an engagement element 78 is provided. FIGS. 24 and 24A disclose a mold 138 for forming an array of conical protuberances 130 as shown and discussed in connection with the embodiment shown in FIGS. 23A-23B. As shown in FIGS. 24 and 24A, the mold includes a base portion 140 and a protuberances portion 142. The protuberances preferably are spaced between 5-500 micrometers apart. In the illustrated embodiment, the mold is capable of making a tissue engaging element which is about 0.175 inch long by about 0.075 inch wide.

In operation, the mold 138 preferably is filled with a resin such as cyanoacrylate, and a vacuum is drawn in order to draw the cyanoacrylate into the protuberance molds. Upon drying, the engaging element can be applied to a harness. The engaging element may be adhered directly to the harness or sutured or otherwise applied. In the embodiment illustrated in FIG. 3, adjacent elastic members are connected by silicone rubber connectors, and tissue engaging elements are adhered to the silicone rubber connectors. In other embodiments, the connectors of the harness are unitarily formed to include protuberances similar to an engaging element. In still other embodiments, a harness can be formed having tissue engaging elements co-formed therewith.

Several other types of materials and prostheses can be used to construct tissue engaging elements. For example, a block of material can be machined to create the element. In other embodiments, relatively large extrusions of material can be cut into several smaller tissue engaging elements. In another preferred embodiment, tissue engaging elements are formed by injection molding. Preferably, the tissue engaging elements are formed of an injection molded polymer, such as urethane. In still another embodiment, tissue engaging elements are constructed of a metal material. During manufacture, the metal is etched electrochemically or otherwise to form surface relief protuberances.

In embodiments discussed above, surface relief protuberances have been depicted as having generally planar surfaces. It is to be understood that, in other embodiments, protuberances having curved, undulating, or even roughened surfaces can be employed.

In the embodiments discussed and illustrated above, aspects of the present invention have been discussed in connection with a cardiac harness embodiment employing elastic rows. In such an embodiment, the harness has an at-rest size that is smaller than the heart, and is elastically deformed to fit the device over the heart. As such, the harness engages the surface of the heart throughout the heart cycle. Also, the harness exerts an inwardly-directed force throughout the heart cycle. This force aids heart function and also forcibly engages the tissue engaging elements with the heart surface. It is to be understood that the aspects discussed above can also be practiced with a cardiac harness having different properties than the illustrated harness. For example, a partially elastic or substantially non-elastic cardiac harness can also benefit from aspects of the embodiments discussed above. In such harnesses, the tissue engaging elements may not be forcibly engaged with the heart surface throughout the entire cardiac cycle. However, the elements will be engaged with the heart surface during at least part of the cycle due to the expansion of the heart and engagement with the harness.

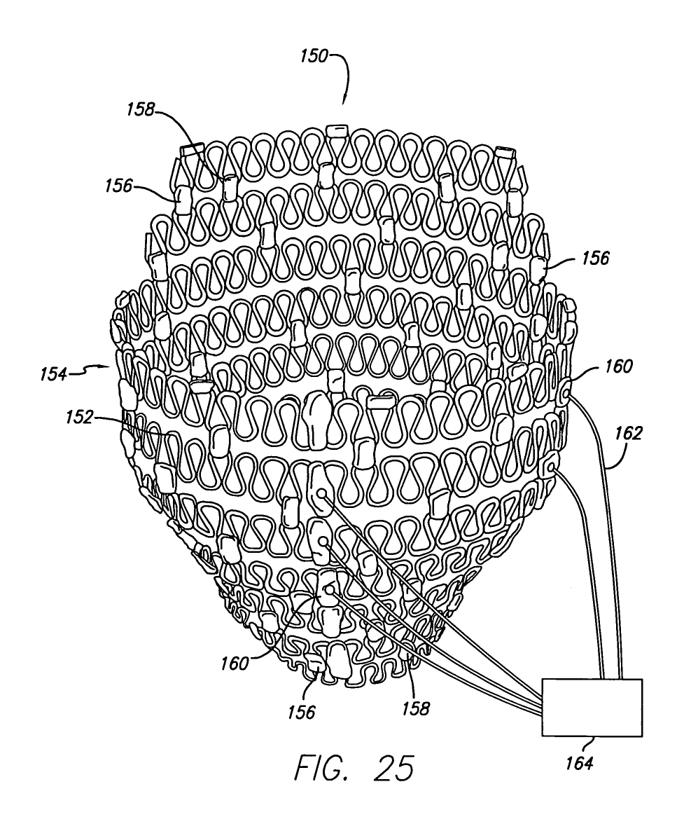

In another embodiment associated with the cardiac harness of the present invention is a cardiac rhythm management device as previously disclosed. Thus, associated with the cardiac harness as shown in FIG. 25, are one or more electrodes for use in providing a defibrillating shock. As can be seen immediately below, any number of factors associated with congestive heart failure can lead to fibrillation, requiring immediate action to save the patient's life.

Diseased hearts often have several maladies. One malady that is not uncommon is irregularity in heartbeat caused by irregularities in the electrical stimulation system of the heart. For example, damage from a cardiac infarction can interrupt the electrical signal of the heart. In some instances, implantable devices, such as pacemakers, help to regulate cardiac rhythm and stimulate heart pumping. A problem with the heart's electrical system can sometimes cause the heart to fibrillate. During fibrillation, the heart does not beat normally, and sometimes does not pump adequately. A cardiac defibrillator can be used to restore the heart to normal beating. An external defibrillator typically includes a pair of electrode paddles applied to the patient's chest. The defibrillator generates an electric field between electrodes. An electric current passes through the patient's heart and stimulates the heart's electrical system to help restore the heart to regular pumping.

Sometimes a patient's heart begins fibrillating during heart surgery or other open-chest surgeries. In such instances, a special type of defibrillating device is used. An open-chest defibrillator includes special electrode paddles that are configured to be applied to the heart on opposite sides of the heart. A strong electric field is created between the paddles, and an electric current passes through the heart to defibrillate the heart and restore the heart to regular pumping.

In some patients that are especially vulnerable to fibrillation, an implantable heart defibrillation device may be used. Typically, an implantable heart defibrillation device includes an implantable cardioverter defibrillator (ICD) or a cardiac resynchronization therapy defibrillator (CRT-D) which usually has only one electrode positioned in the right ventricle, and the return electrode is the defibrillator housing itself, typically implanted in the pectoral region. Alternatively, an implantable device includes two or more electrodes mounted directly on, in or adjacent the heart wall. If the patient's heart begins fibrillating, these electrodes will generate an electric field therebetween in a manner similar to the other defibrillators discussed above.

Testing has indicated that when defibrillating electrodes are applied external to a heart that is surrounded by a device made of electrically conductive material, at least some of the electrical current disbursed by the electrodes is conducted around the heart by the conductive material, rather than through the heart. Thus, the efficacy of defibrillation is reduced. Accordingly, the present invention includes several cardiac harness embodiments that enable defibrillation of the heart and other embodiments disclose means for defibrillating, resynchronization, left ventricular pacing, right ventricular pacing, and biventricular pacing/sensing.

In keeping with the invention, a conductive wire is attached to the coil wire and to a power source. As used herein, the power source can include any of the following, depending upon the particular application of the electrode: a pulse generator; an implantable cardioverter/defibrillator; a pacemaker; and an implantable cardioverter/defibrillator coupled with a pacemaker. In the embodiment shown in FIG. 25, the electrodes are configured to deliver an electrical shock, via the conductive wire and the power source, to the epicardial surface of the heart so that the electrical shock passes through the myocardium.

Commercially available leads having one or more electrodes are available from several sources and may be used with the cardiac harness of the present invention. Commercially available leads with one or more electrodes are available from Guidant Corporation (St. Paul, Minn.), St. Jude Medical (Minneapolis, Minn.) and Medtronic Corporation (Minneapolis, Minn.). Further examples of commercially available cardiac rhythm management devices, including defibrillation and pacing systems available for use in combination with the cardiac harness of the present invention (possibly with some modification) include, the CONTAK CD's®, the INSIGNIA® Plus pacemaker and FLEXTREND® leads, and the VITALITY™ AVT® ICD and ENDOTAK RELIANCE® defibrillation leads, all available from Guidant Corporation (St. Paul, Minn.), and the InSync System available from Medtronic Corporation (Minneapolis, Minn.).

The cardiac rhythm management devices associated with the present invention are implantable devices that provide electrical stimulation to selected chambers of the heart in order to treat disorders of cardiac rhythm and can include pacemakers and implantable cardioverter/defibrillators and/or cardiac resynchronization therapy devices (CRT-D). A pacemaker is a cardiac rhythm management device which paces the heart with timed pacing pulses. As previously described, common conditions for which pacemakers are used is in the treatment of bradycardia (ventricular rate is too slow) and tachycardia (cardiac rhythms are too fast). As used herein, a pacemaker is any cardiac rhythm management device with a pacing functionality, regardless of any other functions it may perform such as the delivery of cardioversion or defibrillation shocks to terminate atrial or ventricular fibrillation. An important feature of the present invention is to provide a cardiac harness having the capability of providing a pacing function in order to treat the dyssynchrony of one or both ventricles. To accomplish the objective, a pacemaker with associated leads and electrodes are associated with and incorporated into the cardiac harness of the present invention. The pacing/sensing electrodes, alone or in combination with defibrillating electrodes, provide treatment to synchronize the ventricles and improve cardiac function.

In one of the preferred embodiments, multi-site pacing using pacing/sensing electrodes enables resynchronization therapy in order to treat the synchrony of both ventricles. Multi-site pacing allows the positioning of the pacing/sensing electrodes to provide bi-ventricular pacing or right ventricular pacing, left ventricular pacing, depending upon the patient's needs.

In further keeping with the invention, some of the tissue engaging elements are formed of a polymer material as previously described, and some of the tissue engaging elements are formed of a metal or metal alloy. As will be described more fully herein, the metal or metal alloy tissue engaging elements are formed having the same basic structure as that described herein for the polymer based tissue engaging elements. The difference, however, is that the metal or metal alloy tissue engaging elements not only provide better frictional engagement to help secure the cardiac harness, but they also can be connected to an internal cardioverter defibrillator (ICD) in order to provide an electrical pulse in the form of a defibrillating shock or for use in pacing/sensing therapy. As described more fully below, the metallic tissue engaging elements are connected via a lead to the ICD so that the tissue engaging elements are in direct contact, preferably with the epicardial surface of the heart, in order to deliver a defibrillating shock or pacing and sensing therapy via the ICD, lead, and tissue engaging elements.

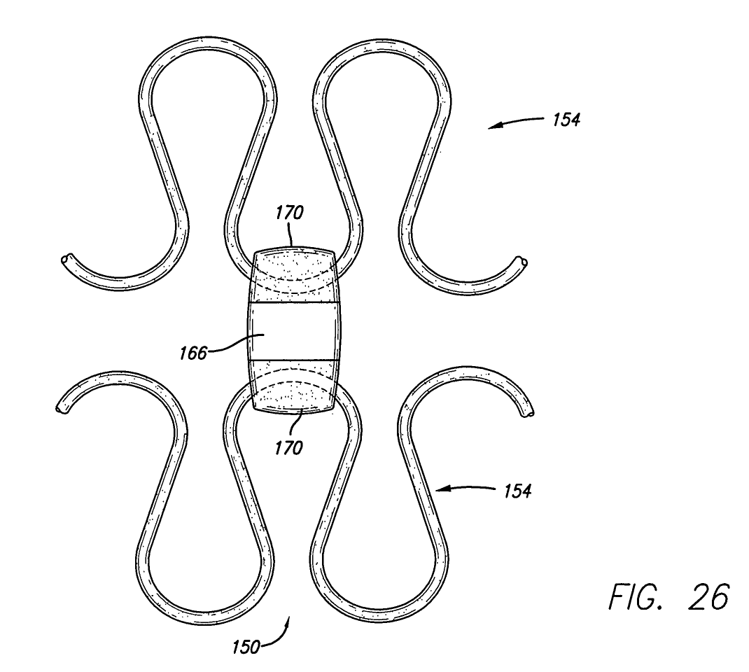

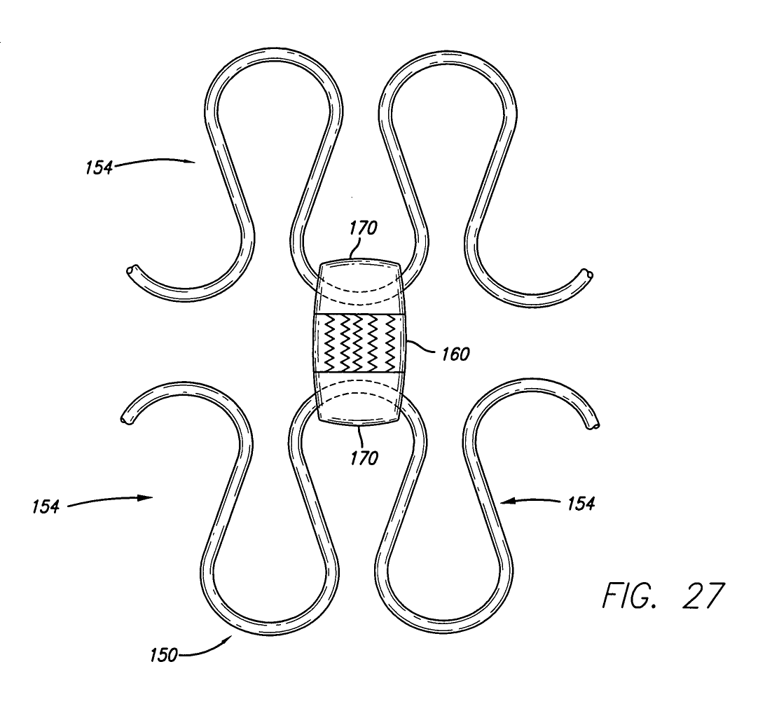

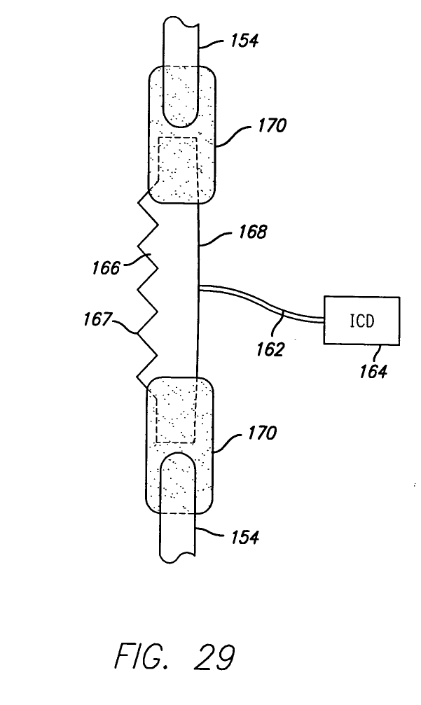

In one embodiment of the invention, shown in FIGS. 25-29, cardiac harness 150 is configured substantially the same as previously described (i.e., FIG. 5). Elastic members 152 are formed in rows 154 to provide an elastic harness capable of applying a compressive force on the heart during at least a portion of the cardiac cycle, and preferably a compressive force during diastole and a slight compressive force during systole. The rows are spaced apart and held together by connectors 156 as previously described. The connectors are in the form of the tissue engaging elements, preferably formed of a polymer, for increasing the frictional engagement between the cardiac harness and the surface of the heart, preferably the epicardial surface of the heart. In this embodiment, some of the tissue engaging elements are formed of a polymer, while other tissue engaging elements are formed of a metal or metal alloy. More specifically, first tissue engaging elements 158 are preferably formed of a polymer as previously described for the purpose of increasing frictional engagement between the cardiac harness and the epicardial surface of the heart. Similarly, second tissue engaging elements 160 also provide enhanced frictional engagement properties, and are formed of a metal or metal alloy. The second tissue engaging elements have the same basic construction as previously described for the first tissue engaging elements made from a polymer. The second tissue engaging elements also are highly conductive and provide a conduit for distributing an electrical shock to the epicardial surface of the heart as will be described.

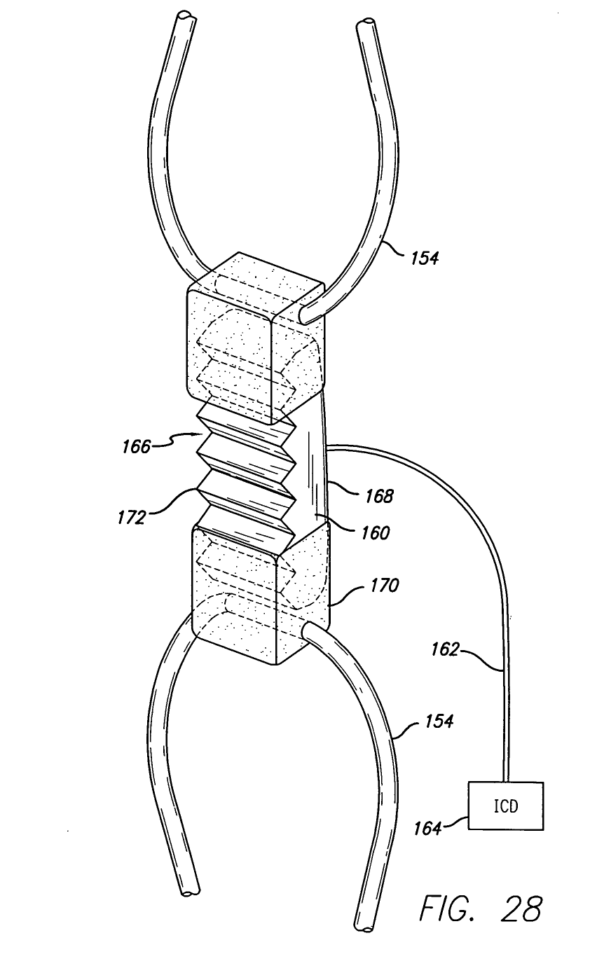

The second tissue engaging elements 160 are connected by leads 162 to an ICD 164. The second tissue engaging elements have a first surface 166 that is in direct contact with the epicardial surface of the heart, and a second surface 168 that is attached to the leads 162. As shown in FIGS. 25-29 the second tissue engaging elements are attached to the rows 154 at interface 170 preferably by a polymer such as silicone rubber or similar dielectric material. It is preferred that the electrical shock delivered by the ICD through the leads and through second tissue engaging elements 160 be insulated from the cardiac harness, which is preferably formed from a super-elastic material such as Nitinol®. The placement of the second tissue engaging elements are a matter of choice and typically would be positioned adjacent the left ventricle and the right ventricle in order to provide a defibrillating shock through the heart. The second tissue engaging elements can be positioned adjacent the left and/or right atria, the left ventricle or the right ventricle, or any combination thereof in order to achieve a particular therapy for each patient. The second tissue engaging elements also can be used for pacing and sensing functions. Since the second tissue engaging elements are in direct contact with the heart they are ideal for sensing cardiac activity, which is relayed through leads 162 to the ICD 164. Further, the second tissue engaging elements can be used for pacing therapies for a particular patient so that a pacing stimulation is delivered by the ICD, through the leads and through the second tissue engaging element to the epicardial surface of the heart. As more clearly shown in FIG. 28, protuberances 172 extend from first surface 166 and may imbed slightly in the epicardial surface of the heart to increase frictional engagement, and also to provide a better conductive path for the defibrillating shock or the pacing/sensing therapy. As previously, described protuberances do not extend into the epicardial a distance far enough to cause injury to the tissue, but only far enough to achieve the dual goals of added frictional engagement and increased contact for delivering an electrical shock.

The size and shape of the second tissue engaging elements 160 is similar to that describe for the tissue engaging elements previously described herein with respect to the polymer first tissue engaging elements. The second tissue engaging elements are formed from a metal or metal alloy which include, but are not limited to gold, platinum, tungsten, stainless steel, Nitinol®, silver, cobalt chromium, titanium, and other biocompatible metals known in the art. Further, the metals or metal alloys that have a high density, such as gold, silver, and the like, also are highly visible under fluoroscopy, so that positioning the second tissue engaging elements adjacent the left ventricle and the right ventricle is more easily accomplished. The second tissue engaging elements 160 can be formed by convention methods which includes, but is not limited to, metal injection molding (MIM), laser cutting, chemical etching, and electrical discharge machinery (EDM). The second tissue engaging elements can then be electropolished or receive other surface finishing treatments.

The foregoing disclosed invention incorporating cardiac rhythm management devices into the cardiac harness combines several treatment modalities that are particularly beneficial to patients suffering from congestive heart failure. The cardiac harness provides a compressive force on the heart thereby relieving wall stress, and improving cardiac function. The defibrillating and pacing/sensing second tissue engaging elements 160 associated with the cardiac harness, along with ICD's and pacemakers, provide numerous treatment options to correct for any number of maladies associated with congestive heart failure. In addition to the defibrillation function previously described, the cardiac rhythm devices can provide electrical pacing stimulation to one or more of the heart chambers to improve the coordination of atrial and/or ventricular contractions, which is referred to as resynchronization therapy. Cardiac resynchronization therapy is pacing stimulation applied to one or more heart chambers, typically the ventricles, in a manner that restores or maintains synchronized bilateral contractions of the atria and/or ventricles thereby improving pumping efficiency. Resynchronization pacing may involve pacing both ventricles in accordance with a synchronized pacing mode. For example, pacing at more than one site (multi-site pacing) at various sites on the epicardial surface of the heart to desynchronize the contraction sequence of a ventricle (or ventricles) may be therapeutic in patients with hypertrophic obstructive cardiomyopathy, where creating asynchronous contractions with multi-site pacing reduces the abnormal hyper-contractile function of the ventricle. Further, resynchronization therapy may be implemented by adding synchronized pacing to the bradycardia pacing mode where paces are delivered to one or more synchronized pacing sites in a defined time relation to one or more sensing and pacing events. An example of synchronized chamber-only pacing is left ventricle only synchronized pacing where the rate in synchronized chambers are the right and left ventricles respectively. Left-ventricle-only pacing may be advantageous where the conduction velocities within the ventricles are such that pacing only the left ventricle results in a more coordinated contraction by the ventricles than by conventional right ventricle pacing or by ventricular pacing. Further, synchronized pacing may be applied to multiple sites of a single chamber, such as the left ventricle, the right ventricle, or both ventricles. The pacemakers associated with the present invention are typically implanted subcutaneously in a patient's chest and have leads attached to the pacing/electrodes as previously described in order to connect the pacemaker to the second tissue engaging elements 160 for sensing and pacing. The pacemakers sense intrinsic cardiac electrical activity through the second tissue engaging elements disposed on the surface of the heart. Pacemakers are well known in the art and any commercially available pacemaker or combination defibrillator/pacemaker can be used in accordance with the present invention.

The cardiac harness and the associated cardiac rhythm management device system of the present invention can be designed to provide left ventricular pacing. In left heart pacing, there is an initial detection of a spontaneous signal, and upon sensing the mechanical contraction of the right and left ventricles. In a heart with normal right heart function, the right mechanical atrio-ventricular delay is monitored to provide the timing between the initial sensing of right atrial activation (known as the P-wave) and right ventricular mechanical contraction. The left heart is controlled to provide pacing which results in left ventricular mechanical contraction in a desired time relation to the right mechanical contraction, e.g., either simultaneous or just preceding the right mechanical contraction. Cardiac output is monitored by impedence measurements and left ventricular pacing is timed to maximize cardiac output. The proper positioning of the pacing/sensing second tissue engaging elements 160 (electrodes) disclosed herein provides the necessary sensing functions and the resulting pacing therapy associated with left ventricular pacing.

An important feature of the present invention is the minimally invasive delivery of the cardiac harness and the cardiac rhythm management device system.

Although the present invention has been described in terms of certain preferred embodiments, other embodiments that are apparent to those of ordinary skill in the art are also within the scope of the invention. Accordingly, the scope of the invention is intended to be defined only by reference to the appended claims. While the dimensions, types of materials and types of engaging elements described herein are intended to define the parameters of the invention, they are by no means limiting and are exemplary embodiments.

I claim:

1. A cardiac harness assembly for treating the heart, comprising:
a self-anchoring cardiac harness having at least a surface for frictionally engaging an outer surface of the heart;
the surface having surface relief protuberances which provide a plurality of tissue engaging elements that apply respective localized forces against the heart without substantially penetrating the heart wall, the tissue engaging elements collectively producing sufficient friction relative to the outer surface so that the harness does not migrate substantially relative to the outer surface; and
the surface relief protuberances being formed from a highly conductive metal and being electrically connected to a power source.

2. The cardiac harness assembly of claim 1, wherein the surface relief protuberances are formed of a material that is less compliant than the heart wall.

3. The cardiac harness assembly of claim 1, wherein the metal is taken from the group of metals consisting of gold, silver, platinum, tungsten, stainless steel, Nitinol, cobalt chromium, and titanium.

4. The cardiac harness assembly of claim 1, wherein the metal includes biocompatible metals and metal alloys.

5. The cardiac harness assembly of claim 1, wherein at least one of the surface relief protuberances is generally pointed.

6. The cardiac harness assembly of claim 5, wherein the pointed protuberance is generally conical.

7. The cardiac harness assembly of claim 5, wherein the pointed protuberance is generally pyramid-shaped.

8. The cardiac harness assembly of claim 1, wherein a first surface relief protuberance comprises an elongate edge.

9. The cardiac harness assembly of claim 8, wherein a second surface relief protuberance comprises an elongate edge that is elongate in a direction transverse to the first elongate edge.

10. The cardiac harness assembly of claim 8, wherein a second surface relief protuberance comprises an elongate edge that is spaced from the first elongate edge.

11. The cardiac harness assembly of claim 1, wherein the surface relief protuberances extend about 10-500 μm.

12. The cardiac harness assembly of claim 10, wherein the surface relief protuberances extend about 10-100 μm.

13. The cardiac harness assembly of claim 1, comprising an engagement element having a plurality of surface relief protuberances

14. The cardiac harness assembly of claim 13, wherein the surface relief protuberances are formed by chemically etching the engagement element.

15. The cardiac harness assembly of claim 13, wherein the surface relief protuberances are formed by metal injection molding.

16. The cardiac harness assembly of claim 13, wherein the surface relief protuberances are formed by laser cutting.

17. The cardiac harness assembly of claim 13, comprising a plurality of spaced apart engagement elements.

18. The cardiac harness assembly of claim 17, wherein surface relief protuberances are disposed only on the engagement elements.

19. The cardiac harness assembly of claim 18, wherein the engagement elements are formed separately from the cardiac harness.

20. The cardiac harness assembly of claim 19, wherein the harness comprises a plurality of rows of elastic material, adjacent ones of the rows being connected by connectors, and engagement elements are disposed on at least some of the connectors.

21. The cardiac harness assembly of claim 20, wherein the engagement elements are electrically insulated from the rows of elastic material.

22. The cardiac harness assembly of claim 21, wherein the engagement elements are attached to leads connected to the power source.

23. The cardiac harness assembly of claim 20, wherein the power source is an implantable cardioverter defibrillator (ICD).

24. The cardiac harness assembly of claim 23, wherein the engagement elements are configured to deliver an electrical shock from the IDC to the heart.

25. The cardiac harness assembly of claim 1, wherein the cardiac harness assembly is configured for minimally invasive delivery.

26. A cardiac harness assembly for treating the heart, comprising:
a cardiac harness having rows connected by first tissue engaging elements;
second tissue engaging elements formed from a biocompatible metal and attached to the rows, the second tissue engaging elements having surface relief protuberances for increasing frictional engagement between the cardiac harness and the heart;
the second tissue engaging elements being connected to leads attached to a power source; and
the second tissue engaging elements being positioned on the cardiac harness so that an electrical shock from the power source transmits through the second tissue engaging elements to deliver a therapeutic electrical shock to the heart.

27. The cardiac harness assembly of claim 26, wherein the second tissue engaging elements are electrically insulated from the rows.

28. The cardiac harness assembly of claim 27, wherein a dielectric material coats the rows and provides an interface connection between the rows and the second tissue engaging elements.

29. The cardiac harness assembly of claim 28, wherein the dielectric material is silicone rubber.

30. The cardiac harness assembly of claim 29, wherein leads extend between the power source and the second tissue engaging elements.

31. The cardiac harness assembly of claim 30, wherein the second tissue engaging elements are formed from a metal or metal alloy taken from the group consisting of gold, silver, platinum, tungsten, stainless steel, Nitinol, cobalt chromium, and titanium.

32. The cardiac harness assembly of claim 31, wherein the surface relief protuberances extend from about 10 to about 50 μm.

33. The cardiac harness assembly of claim 32, wherein the surface relief protuberances extend into the epicardial surface of the heart.

34. The cardiac harness assembly of claim 32, wherein the surface relief protuberances extend onto the epicardial surface of the heart.

35. A method of retaining a cardiac harness on the heart and for providing a therapeutic shock to the heart, comprising:
providing a cardiac harness having a metallic engaging element having surface relief protuberances;
producing friction by pressing the surface relief protuberances on the cardiac harness against a surface of the heart; and
delivering a therapeutic shock from a power source through the metallic engaging element and to the heart.

36. The method of claim 35, wherein no suture is applied to the heart to retain the cardiac harness.

37. The method of claim 35, wherein the surface relief protuberances are adapted to engage the heart surface without substantially penetrating the surface.

38. The method of claim 37, wherein the surface relief protuberances are adapted to engage an epicardial surface of the heart.

39. The method of claim 37 additionally comprising retaining the harness on the heart without substantially penetrating a surface of the heart.

40. The method of claim 35, wherein the cardiac harness is formed from Nitinol and which is configured to apply a compressive force on the heart thereby applying a compressive force on the surface relief protuberances to increase the frictional engagement between the cardiac harness and the heart.

41. The method of claim 40, wherein a defibrillating shock is delivered by the power source through the metallic engaging element to the heart.

42. The method of claim 40, wherein a pacing stimuli is delivered from the power source through the metallic engaging element to the heart.

43. A cardiac harness assembly, comprising:
a cardiac harness for engaging at least a portion of a heart;
a plurality of metallic tissue engaging elements associated with the cardiac harness for increasing the frictional engagement between the cardiac harness and the heart; and
a power source having leads extending between the metallic tissue engaging elements and the power source for delivering a therapeutic shock to the heart.

44. The cardiac harness assembly of claim 43, wherein the tissue engaging elements include surface relief protuberances.

45. The cardiac harness assembly of claim 43, wherein the surface relief protuberances are configured to engage a surface of the heart without substantially penetrating the surface.

46. The cardiac harness assembly of claim 43, wherein the cardiac harness assembly is configured for minimally invasive delivery.

47. The cardiac harness assembly of claim 43, wherein the power source delivers a defibrillating shock through the metallic tissue engaging elements to the heart.

48. The cardiac harness assembly of claim 43, wherein the power source delivers pacing stimuli through the metallic tissue engaging elements to the heart.
